(19) United States
(12) Patent Application Publication
(10) Pub. No.: US 2023/0149488 A1
(43) Pub. Date: May 18, 2023

(54) Compositions and Methods for Cellular Ageing, Stress Resilience, Autophagy, Inflammation and Longevity

(71) Applicant: JUVN3 Holdings, LLC, Millstone, NJ (US)

(72) Inventors: Hector L. Lopez, Carlsbad, CA (US); Tim Ziegenfuss, Carlsbad, CA (US); Matthew Titlow, Carlsbad, CA (US)

(73) Assignees: JUVN3 Holdings, LLC, Millstone, NJ (US); JUVN3 Holdings, LLC, Millstone, NJ (US)

(21) Appl. No.: 16/718,023

(22) Filed: Dec. 17, 2019

Related U.S. Application Data
(60) Provisional application No. 62/849,758, filed on May 17, 2019.
(60) Provisional application No. 62/769,979, filed on Nov. 20, 2018.

(51) Int. Cl. A61K 36/18; A61K 33/30; A61K 33/34; A61K 31/26; A61K 33/04; A61K 31/225; A23L 33/105; A23L 33/15; A23L 33/16

(52) U.S. Cl. CPC A61K 36/18; A61K 33/30; A61K 33/34; A61K 31/26; A61K 33/04; A61K 31/225; A23L 33/105; A23L 33/15; A23L 33/16; A61K 9/0053

(57) ABSTRACT

Compositions and methods are presented that help reduce cellular ageing, improve cellular stress resilience, and/or promote increased longevity. Especially preferred compositions include a combination of a purine alkaloid, an isothiocyanate or thioglucoside, and a metal-containing antioxidant, where the purine alkaloid, the isothiocyanate or thioglucoside, and the metal-containing antioxidant may be chemically isolated compounds or extracts or other preparations from naturally occurring entities such as plants, bacteria, yeast, etc. Advantageously, contemplated compositions will be effective to reduce and repair oxidative stress, improve mitochondrial function, augment DNA repair and telomere maintenance, increase fatty acid metabolism, and modulate histone deacetylation.

This application claims priority to co-pending U.S. Provisional Application No. 62/769,979, filed on Nov. 20, 2018, and U.S. Provisional Application No. 62/849,758, filed on May 17, 2019, the entire contents of both of which are herein incorporated by reference.

### FIELD OF THE INVENTION

The present disclosure relates to compositions and methods of use of various nutraceutical compositions, particularly as they relate to methods of reducing cellular ageing, improving cellular stress resilience, autophagy/mitophagy, functional restoration, cellular regenerative potential, regulating inflammation and/or longevity.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Numerous compositions and methods are known in the art to modulate one or more metabolic parameters in mammals (e.g., insulin or metformin to modulate glucose), however, most commonly such compositions are pharmaceutical agents that often have adverse effects. Moreover, most pharmaceutical compounds are target-specific to a particular receptor or enzyme and as such, provide only isolated effects to a system. Distal effects on other metabolic parameters or pathways are typically unintentional.

In a nutraceutical approach, various compounds are known with pleiotropic effect that can be given in isolation or together with a pharmaceutical or second nutraceutical to increase (in some cases synergistically) a desired effect on a pathway or signaling cascade. For example, quercetin alone, or leucine with low-dose metformin can be administered to stimulate sirtuin pathway output as described in EP 2731599. In another known approach as described in US 2018/0104248, synergistic combinations of theacrine and caffeine or wasabi extract are used to modulate mood, energy, focus, sexual desire, anxiety, or fatigue. While such combination is desirable in at least some instances, such combinations were only effective on a subjective level and were not shown to have substantial impact on one or more aspects of cellular ageing, cellular stress resilience, and/or longevity.

To address at least some aspects of ageing, various telomerase activating compounds were tested and some of them even used in a nutritional supplement as taught in U.S. Pat. No. 8,759,304. Here, selected compounds related to astragalosides and ginsenosides were used to increase telomerase. However, no further pleiotropic effects were reported. On the other hand, increased oxygen consumption rate, extracellular acidification rate, and ATP production was reported in vivo by administering a cold-water extract of humic shale and/or an extract of the apple fruit or skin of the apple fruit as taught in U.S. Pat. No. 9,327,005. Once more, however, while desirable, the biological effects were confined to a relatively narrow effect.

Thus, even though various nutraceuticals are known in the art that provide one or more beneficial effects to cells and organisms, all or almost all of them suffer from various disadvantages. Consequently, there is a need to provide improved compositions and methods that have a wide spectrum of pleiotropic activity, and especially to reduce cellular ageing, improve cellular stress resilience, autophagy/mitophagy, functional restoration, cellular regeneration, regulating inflammation and/or increase longevity.

### SUMMARY OF THE INVENTION

Various compositions and methods that presented that help reduce cellular ageing, improve cellular stress resilience, autophagy/mitophagy, functional restoration, cellular regeneration, regulating inflammation and/or provide increased longevity. Advantageously, such compositions are nutraceutical compositions that comprise pharmaceutically or nutraceutically acceptable ingredients that can be formulated into a variety of formats for oral administration as well as topical administration.

In one aspect of the inventive subject matter, the inventors contemplate a composition for reducing cellular ageing, improving cellular stress resilience, optimizing cellular autophagy/mitophagy, regulating inflammation, and/or increasing longevity that comprises a cytoprotective formulation that includes a combination of (a) a purine alkaloid, (b) an isothiocyanate or thioglucoside, and (c) a metal-containing antioxidant. Most typically, the cytoprotective formulation is formulated for oral administration with a nutritionally or pharmaceutically acceptable carrier.

Therefore, and viewed form a different perspective, the inventors also contemplate a method of reducing cellular ageing and inflammation, improving cellular stress resilience, autophagy/mitophagy and/or increasing longevity in a mammal that includes a step of administering a cytoprotective composition to the mammal in an amount effective to reduce cellular ageing, improve cellular stress resilience, and/or increase longevity in a mammal. Preferably, the cytoprotective composition includes a cytoprotective formulation comprising a combination of (a) a purine alkaloid, (b) an isothiocyanate or thioglucoside, and (c) a metal-containing antioxidant. It is further preferred in such methods that the cytoprotective formulation is formulated for oral administration with a nutritionally or pharmaceutically acceptable carrier.

In another aspect of the inventive subject matter, the inventors contemplate a method of supporting mitochondrial function in a mammal that includes a step of administering a cytoprotective composition to the mammal in an amount effective to support mitochondrial function, wherein the cytoprotective composition includes a cytoprotective formulation comprising a combination of (a) a purine alkaloid, (b) an isothiocyanate or thioglucoside, and (c) a metal-containing antioxidant.

In a further aspect of the inventive subject matter, the inventors contemplate a method of reducing oxidative stress in a cell of a mammal that includes a step of administering a cytoprotective composition to the mammal in an amount effective to reduce oxidative stress, wherein the cytoprotective composition includes a cytoprotective formulation comprising a combination of (a) a purine alkaloid, (b) an isothiocyanate or thioglucoside, and (c) a metal-containing antioxidant.

In yet another aspect of the inventive subject matter, the inventors also contemplate a method of reducing inflammation in a mammal that includes a step of administering a cytoprotective composition to the mammal in an amount effective to reduce inflammation, wherein the cytoprotective composition includes a cytoprotective formulation comprising a combination of (a) a purine alkaloid, (b) an isothiocyanate or thioglucoside, and (c) a metal-containing antioxidant.

Thus, the inventors also contemplate a method of stimulating metabolism in a mammal that has a step of administering a cytoprotective composition to the mammal in an amount effective to stimulate metabolism. Preferably, the cytoprotective composition includes a cytoprotective formulation comprises a combination of (a) a purine alkaloid, (b) an isothiocyanate or thioglucoside, and (c) a metal-containing antioxidant.

For example, in some embodiments, the purine alkaloid is present in and provided as a Camilla sp., Theobroma sp. or Coffea sp. extract, the isothiocyanate or thioglucoside is present in and provided as a Brassica sp. extract, and/or the metal-containing antioxidant comprises copper or zinc as a ligand to an organic moiety. In other embodiments, the purine alkaloid is theacrine, methylliberine, liberine, theobromine, theophylline, or caffeine, the isothiocyanate or thioglucoside is allyl isothiocyanate or 2-phenylethyl isothiocyanate, and/or the metal-containing antioxidant is a copper-(I)-nicotinate complex (or a nutritionally acceptable copper-II-complex or chelate, and less preferably cuprous oxide). In still further embodiments, the purine alkaloid is present in and provided as a Camilla sp., Theobroma sp. or Coffea sp. extract, the isothiocyanate or thioglucoside is present in and provided as a Brassica sp. extract, and the metal-containing antioxidant comprises copper or zinc as a ligand to an organic moiety. In still other embodiments, the purine alkaloid is theacrine, methylliberine, liberine, theobromine, theophylline, or caffeine, the isothiocyanate or thioglucoside is allyl isothiocyanate or 2-phenylethyl isothiocyanate, and the metal-containing is a copper-(I)-nicotinate complex (or a nutritionally acceptable copper-II-complex or chelate). In further preferred embodiments, the purine alkaloid is theacrine (TeaCrine®), the isothiocyanate or thioglucoside is present in and provided as an extract from Eutrema japonicum, and the metal-containing is a copper-(I)-nicotinate complex. Additionally, contemplated compositions may further include fulvate or fulvic acid (and its derivatives), and/or Aronia (e.g., as a powder, expressed juice, or extract).

As will be appreciated, all ingredients can be synthetic, nature-identical, or of natural origin in crude, partially processed, refined, or purified form. For example, purine alkaloids may be synthesized from a precursor, or isolated from a plant part such as a tea leaf, coffee bean and/or coffee fruit. Likewise, the isothiocyanate or thioglucoside may be fully synthetic, or isolated from various plant materials. Moreover, it should be noted that all ingredients may be disposed in a nutritionally acceptable matrix (e.g., within original plant material, fermented material that may or may not include a microorganism) or otherwise suitable carrier. Therefore, contemplated ingredients may be or be derived from natural materials and extracts, recombinant DNA technology, microbial fermentation, total organic synthesis, and any reasonable combination thereof. Of course, it should be appreciated that contemplated compositions may further comprise additional nutritional ingredients, including one or more exogenous gut-supporting and/or ketogenic amplifying compounds (e.g., acetoacetate, beta-hydroxybutyrate, octanoic acid, decanoic acid, tributyrin, butyrate, acetate), SIRT enhancing agents (e.g., butyrate, medium- and short-chain fatty acids, fisetin, resveratrol, quercetin, various catechins, curcumin, tyrosol, berberine, ferulic acid), the non-metal (metalloid) selenium, NAD enhancing agents (e.g., niacinamide, nicotinamide mononucleotide (NMN), or niacin.), one or more phytocannabinoids (e.g., cannabidiol (CBD), cannibigerol, cannabidiolic acid, tetrahydrocannabinol acid), gingerol, shoagol, and/or triterpenes, diterpenes and sesquiterpenes, as well as tea extracts, and especially green or black tea extracts (which may or may not be standardized to have a specific threaflavin and/or thearubigin content).

Notably, the inventive subject matter includes a composition (e.g., a cytoprotective composition) for reducing cellular ageing, improving cellular stress resilience, and/or increasing longevity, wherein the composition includes a cytoprotective formulation including a combination of (a) a purine alkaloid, (b) an isothiocyanate or thioglucoside, and (c) a metal-containing antioxidant; and wherein the cytoprotective formulation is formulated for oral administration with a nutritionally or pharmaceutically acceptable carrier. As disclosed, the purine alkaloid may be present in and provided as a Camilla sp., Theobroma sp., or Coffea sp. extract. Also, the metal-containing antioxidant includes copper or zinc as a ligand to an organic moiety. Additionally, or alternatively, the non-metal (metalloid) selenium may also be included in the composition.

In typical embodiments, the purine alkaloid of the composition for reducing cellular ageing, improving cellular stress resilience, and/or increasing longevity is theacrine, liberine, methylliberine, theobromine, theophylline, or caffeine. In other typical embodiments, the isothiocyanate or thioglucoside is allyl isothiocyanate or 2-phenylethyl isothiocyanate. In still other typical embodiments, the metal-containing antioxidant is a copper-(I)-nicotinate complex or a nutritionally acceptable copper-II-complex or chelate.

Preferably, the contemplated composition for reducing cellular ageing, improving cellular stress resilience, and/or increasing longevity also includes one or more additional nutritional ingredients. Examples of one or more additional nutritional ingredients include a ketogenic compound. Exemplary ketogenic compounds include acetoacetate, tributyrin, beta-hydroxybutyrate (BHB), butyrate, polyhydroxybutyrate (PHB).

The contemplated composition for reducing cellular ageing, improving cellular stress resilience, and/or increasing longevity may also include a SIRT enhancing agent as one or more nutritional ingredients. Exemplary SIRT enhancing agents include fisetin, resveratrol, quercetin, and/or a catechin.

As disclosed in more detail herein, the contemplated composition may include a combination of additional ingredients conferring a combinatorial or even a synergistic effect. Additional nutritional ingredients also include a NAD enhancing agent.

For improved efficacy the contemplated composition for reducing cellular ageing, improving cellular stress resilience, and/or increasing longevity includes a cytoprotective formulation of a combination of (a) a purine alkaloid, (b) an isothiocyanate or thioglucoside, and (c) a metal-containing antioxidant, and (d) additional nutritional ingredients including selenium tributryin, and/or BHB; and wherein the cytoprotective formulation is formulated for oral administration with a nutritionally or pharmaceutically acceptable carrier.

The inventive subject matter also includes methods of reducing cellular ageing, improving cellular stress resilience, and/or increasing longevity in a mammal, in which the mammal is administered the presently contemplated cytoprotective composition as disclosed in various embodiments herein. The method may include administering the cytoprotective composition to the mammal in an amount effective to reduce cellular ageing, inflammation, improve cellular stress resilience, and/or increase longevity in a mammal; wherein the cytoprotective composition includes a cytoprotective formulation comprising a combination of (a) a purine alkaloid, (b) an isothiocyanate or thioglucoside, (c) a metal-containing antioxidant, and optionally (d) one or more additional ingredients including selenium, tributyrin, and/or BHB; wherein the cytoprotective formulation is formulated for oral administration with a nutritionally or pharmaceutically acceptable carrier.

Additionally, the contemplated subject matter includes a method of reducing oxidative stress, autophagy, and/or increasing or maintaining antioxidant activity in a cell of a mammal. This method includes administering a cytoprotective composition to the mammal in an amount effective to reduce oxidative stress; wherein the cytoprotective composition includes a cytoprotective formulation comprising a combination of (a) a purine alkaloid, (b) an isothiocyanate or thioglucoside, (c) a metal-containing antioxidant, and optionally (d) one or more additional ingredients including selenium, tributyrin, and/or BHB.

The inventive subject matter also includes methods and compositions for increasing nicotinamide adenine dinucleotide (NAD+) enzyme activity in a cell of a mammal, including administering the cytoprotective composition as disclosed herein to the mammal in an amount effective to increase increasing nicotinamide adenine dinucleotide (NAD+) enzyme activity. Contemplated methods for increasing NAD+ activity also include inhibiting NAD+ degradation. Typical methods for inhibiting NAD+ degradation include inhibiting CD38 and/or CD157 activity in the cell

Most typically, but not necessarily, the composition may be formulated as a capsule, a tablet, or as a powder, and/or delivers between 25-1,500 mg of the cytoprotective formulation in a single dosage unit. For example, the at least 50 wt % of the composition may be the cytoprotective formulation or a single dosage unit comprises at least 50 mg of the cytoprotective formulation. In other examples, the purine alkaloid may be present in an amount of between 5-500 mg in a single dosage unit, the isothiocyanate or thioglucoside may be present in an amount of between 25-1,000 mg in a single dosage unit, and/or the metal-containing antioxidant may be present in an amount of between 1 mcg-100 mg in a single dosage unit.

Various objects, features, aspects, and advantages will become more apparent from the following detailed description of preferred embodiments, along with the accompanying drawing in which like numerals represent like components.

### BRIEF DESCRIPTION OF THE DRAWING

FIGS. 1A-1F depict exemplary results for mRNA data of SIRT6, SIRT1, NRF2, p27, CDKN2B, ATG12, LCB3, NLRP3, PGC1α, Tomm40, and SIRT4, as indicated, for 3-hour (hr) treatments with Group A, B, C, or D treatments as indicated and as described herein. One-way analysis of variances (ANOVAs) were performed to determine if the treatments significantly differed, and least significant difference (LSD) post hoc tests were performed in order to determine where the significance occurred. Treatments with different superscript letters (a, b, c, or d) indicate treatments differed from each other.

FIGS. 2A-2F depict exemplary results for mRNA data SIRT6, SIRT1, NRF2, p27, CDKN2B, ATG12, LCB3, NLRP3, PGC1α, Tomm40, and SIRT4, as indicated, for 24-hr treatments with Group A, B, C, or D treatments as indicated and as described herein. One-way ANOVAs were performed to determine if treatments significantly differed, and LSD post hoc tests were performed in order to determine where the significance occurred. Treatments with different superscript letters (a, b, c, or d) indicate treatments differed from each other.

FIG. 3 depicts exemplary results for SIRT activity for 3 hour and 24 hour treatments with Group A, B, C, or D, as indicated and as described herein. One-way ANOVAs were performed to determine if treatments significantly differed, and LSD post hoc tests were performed in order to determine where the significance occurred. Treatments with different superscript letters indicates treatments differed from each other.

FIG. 4 depicts exemplary results for citrate synthase activity (mitochondrial capacity marker) following 24 h treatments with Group A, B, C, or D, as indicated and as described herein. One-way ANOVAs were performed to determine if treatments significantly differed, but the ANOVA yielded p=0.367.

FIG. 5A depicts exemplary results for cell viability (DNA/well (ug)) for cells incubated without hydrogen peroxide (CTL, unperturbed), cells incubated with hydrogen peroxide (H2O2), and cells co-incubated with NAD3, BHB, CBD, olive leaf, dynamine, selenium, or tributyrin as indicated.

FIG. 5B depicts exemplary results for total antioxidant capacity (Trolox) for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

FIG. 6A depicts exemplary results for autophagy for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

FIG. 6B depicts exemplary results for DNA damage for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

FIG. 6C depicts exemplary results for cellular oxidative stress for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

FIG. 7A depicts exemplary results for autophagy for cells incubated for 6 hours without hydrogen peroxide (H2O2) (CTL, vehicle (veh) only), and cells incubated with NAD3, BHB, CBD, olive leaf, dynamine, selenium, or tributyrin as indicated.

FIG. 7B depicts exemplary results for quantifying NAMPT protein levels in cells incubated for 6 hours without hydrogen peroxide (H2O2) (CTL, vehicle (veh) only), and cells incubated with NAD3, BHB, CBD, olive leaf, dynamine, selenium, or tributyrin as indicated.

FIG. 7C depicts exemplary results for quantifying PGC1α protein levels in cells incubated for 6 hours without hydrogen peroxide (H2O2) (CTL, vehicle (veh) only), and cells incubated with NAD3, BHB, CBD, olive leaf, dynamine, selenium, or tributyrin as indicated.

FIG. 8A depicts exemplary results for cell viability (DNA/well (ug)) for cells incubated without hydrogen peroxide (CTL, unperturbed), cells incubated with hydrogen peroxide (H2O2), and cells co-incubated with NAD3, NAD3+ CBD, NAD3+olive leaf, NAD3+BHB, NAD3+dynamine, NAD3+selenium, or NAD3+grape seed, as indicated.

FIG. 8B depicts exemplary results for total antioxidant capacity (Trolox) for cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

FIG. 9A depicts exemplary results for autophagy for cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

FIG. 9B depicts exemplary results for DNA damage for cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

FIG. 9C depicts exemplary results for quantifying inflammasome induction by measuring the amount of NLRP3 protein in cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

FIG. 10A depicts exemplary results for autophagy for cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 10B depicts exemplary results for quantifying NAMPT protein levels in cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 10C depicts exemplary results for quantifying PGC1α protein levels in cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 10D depicts exemplary results for quantifying total SIRT activity in cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 11A depicts representative protein gels of C2C12 (muscle cells) with Western blotting analysis shown for p-H2AX, LC3I, LC3II, 4HNE, and NLRP3, and Ponceau protein staining, as indicated.

FIG. 11B depicts representative protein gels of EOMA (endothelial cells) with Western blotting analysis shown for p-H2AX, LC3I, LC3II, 4HNE, and NLRP3, and Ponceau protein staining, as indicated.

### DETAILED DESCRIPTION

The inventors have now discovered that nutraceutical compositions can be prepared that have significant impact on cellular ageing, cellular stress resilience, longevity, and pathways and pathway elements associated therewith. In especially preferred aspects, contemplated compositions will include a cytoprotective formulation that comprises a combination of (a) a purine alkaloid, (b) an isothiocyanate or thioglucoside, and (c) a metal-containing antioxidant, where such ingredients can be isolated and/or purified (e.g., having chemical purity of at least 90 mol %) or where such ingredients can be present or be provided in form of an extract or other preparation from a plant, yeast, or animal.

For example, in some preferred embodiments, the purine alkaloid is present in and provided as a Camilla sp., Theobroma sp., or Coffea sp. extract, or extract from the Cupuaçu plant, or seeds of Herrania species, while in other preferred embodiments the purine alkaloid is theacrine, theobromine, theophylline, or caffeine. Likewise, in some preferred embodiments the isothiocyanate or thioglucoside is present in and provided as a Brassica sp. extract (e.g., wasabi extract), while in other preferred embodiments the isothiocyanate or thioglucoside is allyl isothiocyanate or 2-phenylethyl isothiocyanate. Furthermore, in some preferred embodiments, the metal-containing antioxidant comprises copper (or other transition metal) or zinc as a ligand to an organic moiety, while in further preferred embodiments the metal-containing antioxidant is a copper-(I)-nicotinate complex (of course, in alternative embodiments copper-II-complexes or chelates with orotate, amino acids, etc. and even colloidal copper are also deemed appropriate).

Thus, and as is described in more detail below, one exemplary composition for reducing cellular ageing, improving cellular stress resilience, and/or increasing longevity will include a cytoprotective formulation comprising a combination of (a) theacrine, (b) a wasabi extract as source of an isothiocyanate or thioglucoside, and (c) a copper-(I)-nicotinate complex as antioxidant. Where theacrine is used, various sources may be employed. However, a particularly preferred form of theacrine is TeaCrine™ (generic name theacrine, commercially available). As noted herein, all ingredients can be synthetic, nature-identical, or of natural origin in crude, partially processed, refined, or purified form. For example, purine alkaloids may be synthesized from a precursor, or isolated from a plant part such as a tea leaf, coffee bean and/or coffee fruit. Likewise, the isothiocyanate or thioglucoside may be fully synthetic, or isolated from various plant materials. Moreover, it should be noted that all ingredients may be disposed in a nutritionally acceptable matrix (e.g., within original plant material, fermented material that may or may not include a microorganism) or otherwise suitable carrier. Therefore, contemplated ingredients may be or be derived from natural materials and extracts, recombinant DNA technology, microbial fermentation, total organic synthesis, and any reasonable combination thereof.

Based on experimental findings and data as presented in more detail below, the components of the cytoprotective formulation are deemed to have multiple desirable and pleiotropic effects that address multiple biological systems. Particularly, contemplated compositions and methods are thought to support and improve mitochondrial function and integrity (e.g., via SIRT1, SIRT4, global SIRT1-7 activity, PGC-1α, TOMM40 upregulation), provide anti-inflammatory effects and immunomodulation (e.g., by NLRP3/inflammasome downregulation), stimulate longevity gene expression/activity (e.g., by upregulation of SIRT proteins), optimize NAD metabolic pathways to increase energy metabolism (e.g., via upregulation of PGC-1α), improve DNA repair processes (e.g., via SIRT1 and SIRT6 upregulation), and/or increase telomere stability/length (e.g., via SIRT1 and SIRT6 upregulation).

More particularly, contemplated compositions and methods will advantageously affect one or more physiological markers that are associated with pathways regulating mitochondrial integrity and function, inflammation and cellular stress response, energy metabolism, and especially fatty acid oxidation, longevity, DNA repair, and/or telomere maintenance/lengthening. For example, and as described in more detail below, contemplated compositions are advantageously capable to modulate various stress response mechanisms and pathways that control cytointegrity. Among other pathways, contemplated compositions may affect stress response proteins such as Hsp70, Hsp16, Hsp90, and/or SOD3 as well as Wnt/beta-catenin or Lin-44/Wnt signaling pathways, ageing related pathways that include ELT-3 transcription factors, or may downregulate pathways that include Mom-2/Wnt or Cwn-2/Wnt signaling. Therefore, and viewed from a system perspective, contemplated compositions will beneficially regulate autophagy and/or mitophagy, increase stress resistance and stress response, help maintain DNA integrity and repair, and promote telomere integrity, all of which are deemed hallmarks of longevity. Moreover, due to the pleiotropic nature of the compositions presented herein, signaling pathways are affected that interact with one another and so provide a multi-mechanistic cytoprotective effect that drives cellular metabolism and repair and stress response towards a profile associated with health and longevity. Such effects are believed to operate on a cellular level, tissue level, and even systemic level.

In a first embodiment, the marker is SIRT1, which is a NAD-dependent protein deacetylase that links transcriptional regulation directly to intracellular energetics and is also known to participate in the coordination of several separated cellular functions, including cell cycle, response to DNA damage, metabolism, apoptosis, and autophagy. Moreover, SIRT1 can modulate chromatin function through deacetylation of histones and can thereby promote alterations in the methylation of histones and DNA, leading to transcriptional repression of genes affected by methylation or other epigenetic changes. In addition, SIRT1 is known to deacetylate a broad range of transcription factors and co-regulators, thereby regulating target gene expression positively and negatively. For example, SIRT1 was shown to de-acetylate and affect the activity of both members of the PGC1-alpha/ERR-alpha complex, which are essential metabolic regulatory transcription factors.

With regard to SIRT1 function on energy metabolism it should be appreciated that SIRT1 serves as a sensor of the cytosolic ratio of NAD(+)/NADH, which is altered by glucose deprivation and metabolic changes associated with caloric restriction. Moreover, SIRT1 is also a component of the eNoSC (energy-dependent nucleolar silencing complex), a complex that mediates silencing of rDNA in response to intracellular energy status and acts by recruiting histone-modifying enzymes. The eNoSC complex is able to sense the energy status of cell: upon glucose starvation, elevation of NAD(+)/NADP(+) ratio activates SIRT1

With regard to DNA repair it was proposed that SIRT1 also contributes to genomic integrity via positive regulation of telomere length, and that SIRT1 is involved in DNA damage response by repressing genes which are involved in DNA repair, such as XPC and TP73, deacetylating XRCC6/Ku70, and facilitating recruitment of additional factors to sites of damaged DNA. For example, SIRT1-deacetylated NBN can recruit ATM to initiate DNA repair and SIRT1-deacetylated XPA can interacts with RPA2. Moreover, SIRT1 also deacetylates WRN, thereby regulating its helicase and exonuclease activities and regulates WRN nuclear translocation in response to DNA damage. Additionally, SIRT1 deacetylates APEX1 and stimulates cellular AP (apurinic site) endonuclease activity by promoting the association of APEX1 to XRCC1. Finally, SIRT1 is also thought to deacetylate XRCC6/Ku70 at Lys-539 and Lys-542 causing it to sequester BAX away from mitochondria thereby inhibiting stress-induced apoptosis.

In a second embodiment, the marker is SIRT4, which is typically located in the cytoplasm, mitochondria, and nucleus of a cell and which is ubiquitously expressed. SIRT4 is thought to have multiple catalytic functions and was reported to operate as an NAD-dependent protein lipoamidase, an ADP-ribosyl transferase, and a deacetylase. In most cases, SIRT4 catalyzes more efficiently removal of lipoyl- and biotinyl- than acetyl-lysine modifications. As a consequence, the pyruvate dehydrogenase complex (PDH) activity can be inhibited via the enzymatic hydrolysis of the lipoamide cofactor from the E2 component, DLAT, in a phosphorylation-independent manner. SIRT4 also catalyzes the transfer of ADP-ribosyl groups onto target proteins, including mitochondrial GLUD1 (glutamate dehydrogenase 1), inhibiting GLUD1 enzyme activity. As such, SIRT4 can act as a negative regulator of mitochondrial glutamine metabolism by mediating mono ADP-ribosylation of GLUD1: expressed in response to DNA damage and negatively regulates anaplerosis by inhibiting GLUD1, leading to block metabolism of glutamine into tricarboxylic acid cycle and promoting cell cycle arrest. In response to mTORC1 signal, SIRT4 expression is repressed, promoting anaplerosis and cell proliferation. Moreover, SIRT4 also acts as a NAD-dependent protein deacetylase, mediating deacetylation of Lys-471 of MLYCD, inhibiting its activity, thereby acting as a regulator of lipid homeostasis. Finally, SIRT4 was also reported to control fatty acid oxidation by inhibiting PPARA transcriptional activation.

In a third embodiment, the marker is SIRT6. As the name implies, SIRT6 is a member of the sirtuin family of NAD-dependent enzymes that are implicated in cellular stress resistance, genomic stability, aging and energy homeostasis. SIRT6 is localized to the nucleus, exhibits ADP-ribosyl transferase and histone deacetylase activities, and plays a role in DNA repair, maintenance of telomeric chromatin, inflammation, lipid and glucose metabolism. Moreover, SIRT6 was also reported as a stress responsive protein deacetylase and as a mono-ADP ribosyltransferase.

With regard to DNA repair, SIRT6 was reported as a chromatin-associated protein that is required for normal base excision repair of DNA damage in mammalian cells. Moreover, SIRT6 has deacetylase activity towards histone H3K9Ac and H3K56Ac and modulates acetylation of histone H3 in telomeric chromatin during the S-phase of the cell cycle. Notably, SIRT6 was alo reported to deacetylate histone H3K9Ac at NF-kappa-B target promoters and may therefore down-regulate the expression of a subset of NF-kappa-B target genes. Additionally, SIRT6 may also act as a corepressor of the transcription factor HIF1A to so control expression of multiple glycolytic genes to regulate glucose homeostasis, which has substantial impact on energy metabolism.

Therefore, on a functional level, SIRT6 can be viewed as a required factor for genomic stability, as a regulator for the production TNF, and as a modulator of cellular senescence and apoptosis.

In a fourth embodiment, the marker is NRF2 (nuclear factor (erythroid-derived 2)-like 2), that is known to regulate the expression of various antioxidant proteins that protect against oxidative damage triggered by injury and inflammation. As such the systemic and local function of NRF2 is subastantial in cytoprotection against oxidative stress and inflammatory mediator signals and inflammation. Mechanistically, NRF2 acts as a transcription activator that binds to antioxidant response (ARE) elements in the promoter regions of target genes with such elements. Consequently, NRF2 is a critical mediator for coordinated up-regulation of genes required for response to oxidative stress.

In a fifth embodiment, the marker is p27 (cyclin-dependent kinase inhibitor 1B) that acts as a regulator of cell cycle progression. More specifically p27 is thought to inhibit the kinase activity of CDK2 bound to cyclin A, but thought to have little inhibitory activity on CDK2 bound to SPDYA. P27 is also a potent inhibitor of cyclin E- and cyclin A-CDK2 complexes, forms a complex with cyclin type D-CDK4 complexes, and is involved in the assembly, stability, and modulation of CCND1-CDK4 complex activation. Notably, p27 can act either as an inhibitor or an activator of cyclin type D-CDK4 complexes, depending on its phosphorylation state and/or stoichiometry. On a functional level, p27 is considered a tumor suppressor because of its function as a regulator of the cell cycle, and as such helps maintain cellular homeostasis and integrity.

In a sixth embodiment, the marker is CDKN2B/p15. This protein acts as a cyclin-dependent kinase 4 inhibitor B (cyclin-dependent kinase inhibitor), which forms a complex with CDK4 or CDK6, and so prevents the activation of the CDK kinases. Consequently, and on a functional level, CDKN2B functions as a cell growth regulator that controls cell cycle G1 progression, and as such helps maintain cellular homeostasis and integrity.

In a seventh embodiment, the marker is ATG12 (also known as autophagy-related protein 12). ATG12 is a ubiquitin-like protein that is involved in autophagy vesicle formation. Autophagy is a process of bulk protein degradation in which cytoplasmic components, including organelles, are enclosed in double-membrane structures called autophagosomes and delivered to lysosomes or vacuoles for degradation. Conjugation with ATG5 through a ubiquitin-like conjugating system involving also ATG7 as an E1-like activating enzyme (ATG10 as an E2-like conjugating enzyme is essential for its function). The ATG12-ATG5 conjugate acts as an E3-like enzyme which is required for lipidation of ATG8 family proteins and their association to the vesicle membranes. Thus, ATG12 is involved in cell maintenance, support of cellular integrity, and protein turnover. Moreover, ATG12 conjugation to ATG3 is also reported to help regulate mitochondrial homeostasis (possibly through interaction with Bcl-2) and cell death. In that context, further suitable markers include those related to regulation of mitophagy/autophagy, and especially transcription factors, co-activators, and regulatory proteins such as HMGB1, BNIP3, NIX, ACAA2, GABARAPL1, etc.

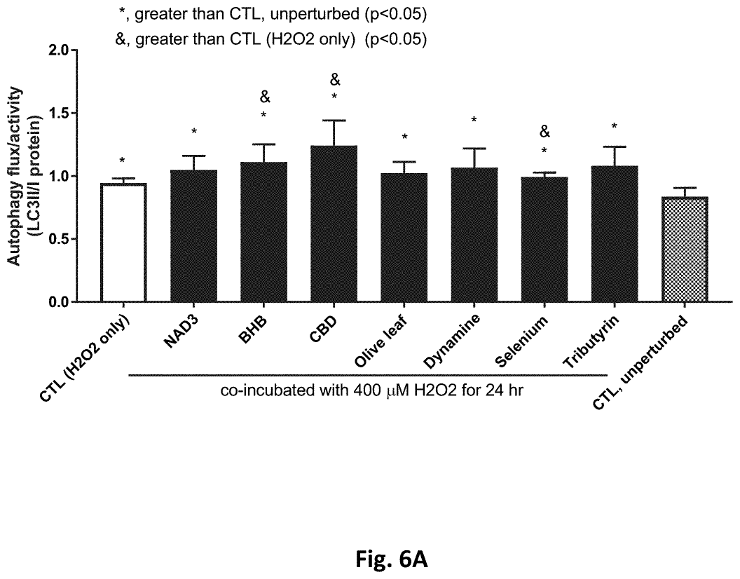

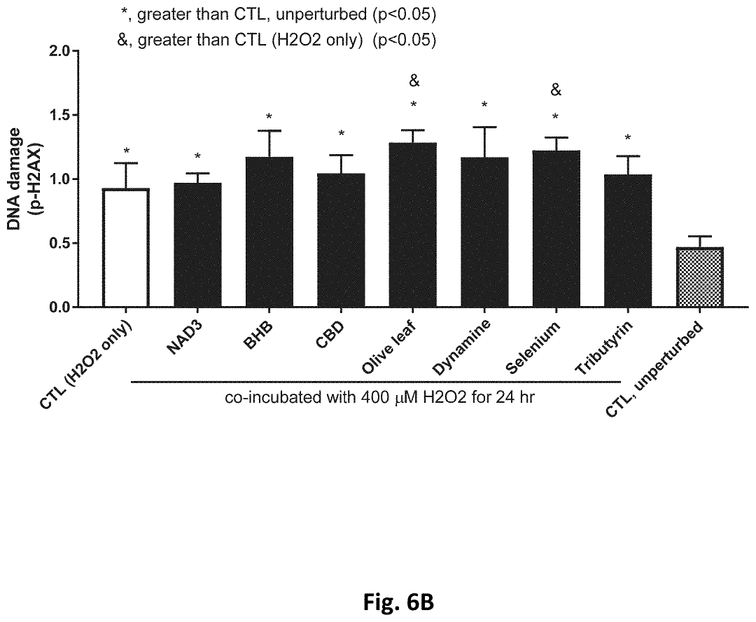

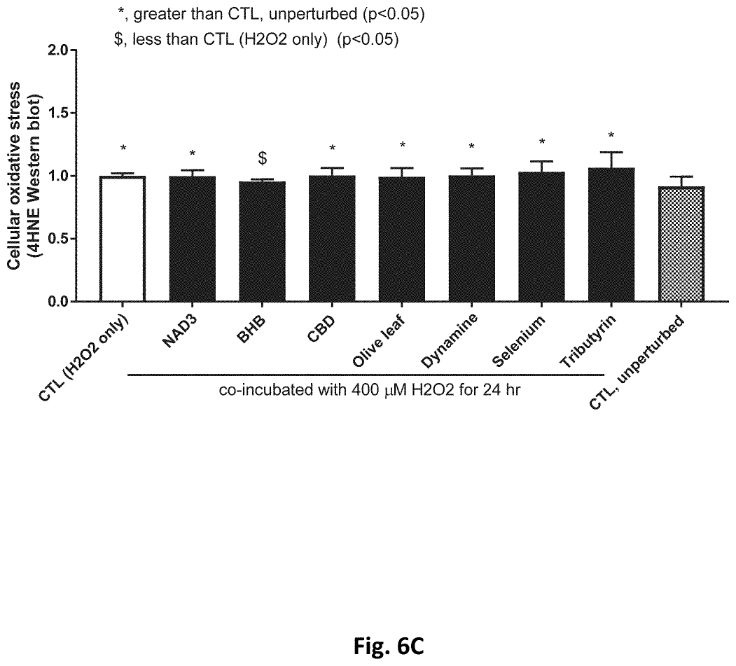

In an eighth embodiment, the marker is LCB3 (SPTLC3): LCB3 is a serine palmitoyltransferase, and the heterodimer formed with LCB1/SPTLC1 constitutes the catalytic core. The composition of the serine palmitoyltransferase (SPT) complex determines the substrate preference. The SPTLC1-SPTLC3-SPTSSA isozyme uses both C₁₄-CoA and C₁₆-CoA as substrates, while the SPTLC1-SPTLC3-SPTSSB has the ability to use a broader range of acyl-CoAs without apparent preference. As such, LCB3 is an important regulator in sphingolipid metabolism. Notably, sphingolipid metabolites, such as ceramide and sphingosine-1-phosphate, have been shown to be important mediators in the signaling cascades involved in apoptosis, proliferation, stress responses, necrosis, inflammation, autophagy, senescence, and differentiation. Consequently, LCB3 is a substantial factor in pathways regulating stress response, autophagy, and senescence. With reference to FIG. 6A, increased autophagy was observed in cells incubated with beta hydroxybutyrate (BHB), cannabidiol (CBD), or selenium.

In a ninth embodiment, the marker is NLRP3 (cryoporin). NLRP is traditionally viewed as a PRP (pathogen recognition receptor) and is predominantly expressed in macrophages. Moreover, NLRP3 is also a component of the inflammasome, and detects products of damaged cells such as extracellular ATP. As the sensor component of the NLRP3 inflammasome, NLRP3 plays a critical role in innate immunity and inflammation. In response to pathogens and other damage-associated signals, NLRP3 initiates the formation of the inflammasome polymeric complex, made of NLRP3, PYCARD and CASP1 (and possibly CASP4 and CASP5). Recruitment of proCASP1 to the inflammasome promotes its activation and CASP1-catalyzed IL1B and IL18 maturation and secretion in the extracellular milieu. Inflammasomes can also induce pyroptosis, an inflammatory form of programmed cell death. Under resting conditions, NLRP3 is autoinhibited. NLRP3 activation stimuli include extracellular ATP, reactive oxygen species, K(+) efflux, crystals of monosodium urate or cholesterol, amyloid-beta fibers, environmental or industrial particles and nanoparticles, cytosolic dsRNA, etc. Independently of inflammasome activation, regulates the differentiation of T helper 2 (Th2) cells and has a role in Th2 cell-dependent asthma and tumor growth. Consequently, NLRP3 is an important player in stress response of a cell to cell damage and oxidative stress.

In a tenth embodiment, the marker is PGC-1α(peroxisome proliferator-activated receptor gamma coactivator 1-alpha). PGC-1α operates as a transcriptional coactivator for steroid receptors and nuclear receptors and as such increases the transcriptional activity of PPARG and thyroid hormone receptor on the uncoupling protein promoter. Moreover, PGC-1α can regulate key mitochondrial genes that contribute to the program of adaptive thermogenesis and further plays an essential role in metabolic reprogramming in response to dietary availability through coordination of the expression of a wide array of genes involved in glucose and fatty acid metabolism. Therefore, PGC-1α is a significant marker for regulation of genes involved in energy metabolism (and may be viewed as master regulator of mitochondrial biogenesis). Notably, PGC-1α is also thought to be involved in the integration of the circadian rhythms, and with that in energy metabolism (as PGC-1α is required for oscillatory expression of clock genes, such as ARNTL/BMAL1 and NR1D1).

In an eleventh embodiment, the marker is TOMM40 (translocase of outer mitochondrial membrane 40 homolog (yeast)). TOMM40 is a protein that is localized in the outer membrane of the mitochondria and is the channel-forming subunit of the translocase of the mitochondrial outer membrane (TOM) complex that is essential for import of protein precursors into mitochondria. Thus, TOMM40 may be used as a suitable marker for mitochondrial function and health.

Of course, it should be appreciated that the contemplated compositions and methods presented herein will advantageously affect not only one marker as noted above, but may modulate two, three, four, five, six, seven, eight, nine, ten, or all of the markers noted above, possibly in a synergistic manner to so exert the pleiotropic effect.

Consequently, contemplated compositions will be formulated to affect not only one marker as noted above, but may modulate two, three, four, five, six, seven, eight, nine, ten, or all of the markers noted above, possibly in a synergistic manner. Thus, multiple pathways can be addressed to achieve (preferably synergistic) effects with regard to at least one of mitochondrial integrity and function, inflammation and cellular stress response, energy metabolism, and especially fatty acid oxidation, longevity, DNA repair, and/or telomere maintenance/lengthening.

To that end the inventors contemplate that a composition will include a cytoprotective formulation that comprises a combination of (a) a purine alkaloid, (b) an isothiocyanate or thioglucoside, and (c) a metal-containing antioxidant, preferably in quantities and proportions that are effective to improve at least one of mitochondrial integrity and function, inflammation and cellular stress response, energy metabolism, and especially fatty acid oxidation, autophagy/mitophagy, regenerative stem cell potential, longevity, DNA repair, and/or telomere maintenance/lengthening.

For example, one exemplary composition will include theacrine (e.g., purity at least 90 mol %, and more preferably at least 95 mol %), a wasabi extract (preferably standardized to isothiocyanates) as a source of isothiocyanates or thioglucosides, and cuprous nicotinate as the a metal-containing antioxidant. In further preferred examples, the composition is formulated for oral administration (e.g., as a capsule or tablet as dosage unit) and includes theacrine in an amount of between 10-500 mg in a single dosage unit, the wasabi extract in an amount of between 50-1,000 mg in a single dosage unit, and the cuprous nicotinate in an amount of between 1 mcg-100 mg in a single dosage unit.

The inventors also contemplate a composition that includes a cytoprotective formulation comprising a combination of a purine alkaloid, an isothiocyanate or thioglucoside, a metal containing antioxidant, nicotinamide riboside alternative (NAD3), and one or more of the following: the non-metal or metalloid selenium (Se), tributyrin, beta hydroxybutyrate (BHB), beta hydroxybutyric acid, butyrate, poly hydroxybutyrate (PHB), humic shale extracts, fulvate or fulvic acid (and its derivatives such as those described in US2016/0066603 or U.S. Pat. No. 6,440,436), triproprionin, triacetin, palmitoleic acid, and/or gamma linolenic acid (GLA). The inventors have surprisingly found that the addition of tributyrin and NAD3 in the composition of a purine alkaloid, an isothiocyanate or thioglucoside, and a metal containing antioxidant gives additional beneficial effect with regards to inflammation and aging. Tributyrin helps create tighter junctions in the colon epithelium leading to less gut waste circulating in the blood and less systemic inflammation, which in turn leads to reduced aging. The inventors surprisingly found that this particular composition has a synergistic effect and is ideal for the gut-brain-heart vagus nerve trifecta as Cu(I) improves heart health and theacrine acts on the brain cells to improve mood, energy, and focus.

As shown in exemplary results in the present disclosure (e.g., Figs.)—notably, increased levels of NAMPT protein and decreased induction of NLRP3, the presently disclosed methods and compositions support nicotinamide adenine dinucleotide (NAD+) enzyme activity. Accordingly, contemplated methods and compositions for reducing cellular ageing, improving cellular stress resilience, and/or increasing longevity include the disclosed cytoprotective composition having a cytoprotective formulation including a combination of (a) a purine alkaloid, (b) an isothiocyanate or thioglucoside, (c) a metal-containing antioxidant, and optionally (d) one or more additional ingredients including selenium, a polyphenol, tributyrin, and/or BHB. In addition to methods and compositions which increase NAD+, the inventive subject matter also includes decreasing NAD+ degradation. Typically, cytoprotective methods and compositions inhibit NAD+ degradation by inhibiting CD38 and/or CD157 activity. See, e.g., Wang et al., 2019, Front Physiol., 10:1-10 and Ruan et al., 2018, Pharmacol Res. 128:345-358.

In addition to tributyrin, butyrate, BHB, and PHB, other fatty acids are contemplated to be present in the composition as well. Examples of such fatty acids are triproprionin and triacetin. Furthermore, a short chain fatty acid such as palmitoleic acid or gamma linolenic acid (GLA), may improve skin conditions such as eczema, atopic dermatitis.

However, it should be appreciated that numerous alternative compounds may be included in the cytoprotective formulation in addition to or as an alternative to the above compounds. For example, theacrine may be replaced by (or supplemented with) one or more theacrine prodrug, a theacrine metabolite, and/or a theacrine analogs. For example such compounds include liberine or methylliberine, caffeine, methylated or acetylated theacrine, etc. Likewise, the wasabi extract may be replaced or supplemented with various alternative extracts or preparations (e.g., dried, powderized, etc.) of portions of a plant or sprout belonging to the family Brassicaceae (such as Armoracia rusticana). Other suitable plant preparations include Aronia, which may be in form of expressed juice (which may be further processed), a dried powder, or in form of an extract. Alternatively, or additionally, the isothiocyanate or thioglucoside may also be a chemically isolated or synthetic isothiocyanate or thioglucoside. For example, suitable isothiocyanates especially include allyl isothiocyanate and or 2-phenylethyl isothiocyanate. Of course, it should be appreciated that suitable compounds may also be present as precursors, most typically those cleavable by myrosinase such as various thioglucosides (esp. gluosinolates). Likewise, the metal-containing antioxidant need not be limited to a copper-(I)-nicotinate complex, but all nutritionally acceptable forms of copper such as copper-I/II-complexes and chelates (e.g., defined complexes with orotate, amino acids, etc., or undefined as in a complex food matrix (e.g., U.S. Pat. No. 8,642,651)) are also expressly contemplated. Moreover, various other antioxidants containing a (transition) metal portion are deemed suitable for use herein (see e.g., Curr Top Med Chem. 2011; 11(21):2703-13). Especially suitable metal antioxidants will not only provide antioxidant capacity but also deliver desirable quantities of the metal to the mitochondria in a cell and/or to various enzymes (such as SIRT) as a co-factor. Thus, contemplated (transition) metals especially include copper, zinc, iron, and manganese. In this context, it should be noted that copper, zinc, manganese, and other metals may be co-administered (or even replace cuprous nicotinate), and administration of such metals will typically follow known dosages and quantities.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.).

In typical embodiments, a cytoprotective composition for oral or topical administration is disclosed herein wherein the composition includes a cytoprotective formulation comprising a combination of (a) a purine alkaloid, (b) an isothiocyanate or thioglucoside, (c) a metal-containing antioxidant, and optionally (d) one or more additional ingredients including selenium, a polyphenol, tributyrin, and/or BHB.

For topical administration, the inventive subject matter includes topical (e.g., nutracosmeceutical) cytoprotective compositions. In general, compositions for topical and oral administration are understood by those skilled in the art. For example, NAD3 enhancement compositions for skin and skin cancer therapy as well as topical therapies for inflammasome activation are known in the art. See, e.g., Garcia-Peterson et al., 2017, Skin Pharmacol Physiol., 30:216-224 and Rong-Jane Chen et al., 2016, Int. J. Mol. Sci, 17:1-16.

Most typically, contemplated compositions will deliver between 10-500 mg, or between 10-800 mg, or between 20-1,000 mg, or between 30-1,200 mg, or between 50-1,500 mg, or between 100-2,000 or even more of the cytoprotective formulation in a single dosage unit. Viewed from a different perspective, at least 20 wt %, or at least 30 wt %, or at least 40 wt %, or at least 50 wt %, or at least 60 wt %, or at least 70 wt %, or at least 80 wt %, or at least 90 wt % of the composition will be the cytoprotective formulation. Consequently, preferred oral single dosage units (or recommended daily uptake) will be between 20-200 mg, or between 40-400 mg, or between 60-600 mg, or between 80-800 mg, or between 100-1,000 mg, or between 200-2,000 mg, and in some cases even higher.

Preferably, the purine alkaloid is present in an amount of between 10-400 mg, such as between 10-100 mg, or between 20-200 mg, or between 30-300 mg, or between 40-400 mg, or between 50-500 mg, or between 50-750 mg, or between 100-1,000 mg, or between 200-2,000 mg in a single dosage unit. Likewise, the isothiocyanate or thioglucoside is present in an amount of between 10-150 mg, or between 20-400 mg, or between 50-750 mg, or between 75-1,000 mg, or between 100-1,500 mg, or between 250-2,500 mg, in a single dosage unit where an extract or plant preparation is used. Where isolated isothiocyanates or thioglucosides are used, the isothiocyanate or thioglucoside is present in an amount of between 1-15 mg, or between 2-40 mg, or between 5-75 mg, or between 10-100 mg, or between 20-150 mg, or between 50-250 mg, or even higher in a single dosage unit. Depending on the type of metal-containing antioxidant, the metal-containing antioxidant may be present in an amount of between 1 mcg-1 mg, or between 10 mcg-100 mcg, or between 100 mcg to 1 mg, or between 1-20 mg, or between 5-75 mg, or between 10-100 mg, or between 20-150 mg, or between 50-250 mg, or even higher in a single dosage unit.

Moreover, it should be appreciated that contemplated oral and topical compositions may further include additional functional ingredients that may provide further enhancement of the desired effects as presented herein. For example, suitable longevity enhancing ingredients include acetoacetate, and beta-hydroxybutryate, while compounds that contribute to enhancing SIRTs and SIRT Targets include trans-resveratrol, quercetin, honokiol, oroxylin-A, EGCG, berberine, hydroxy-tyrosol (all of which cross-modulate targets that help preserve mitochondrial function, metabolic flexibility, endothelial/vascular function, while inhibiting systemic inflammation, apoptosis, and fibrosis). Additional ingredients include berberine, curcumine, various stilbenoids such as picetannol, one or more chalcones, catechins, and flavonols such as butein phytocannabinoids and NAD precursors. Moreover, suitable additional ingredients may also be added as absorption enhancers and therefore include various vanilloids, piperine, ascorbic acid, etc. Where desired, additional ingredients also include various enzymes, and particularly those that support antioxidant systems. Therefore, superoxide dismutase is contemplated as an exemplary additional ingredient.

Therefore, additional ingredients besides the compounds in the cytoprotective formulation include more bioavailability enhancers, including for example polyphenols, bioperine, piperine, black pepper, bergamottin, dihydroxybergamottin (CYP3A4 inhibitors), flavonoids (including hesperidin, naringin, tangeritin, quercetin and nobiletin both in isolation and in combination), turmeric, triterpenoids (e.g., beta-caryophyllene, d-limonene, limonol, myrcene, etc.), pterostilbenes, fisetin. Suitable analgesics and anti-inflammatory agents include ibuprofen, salicylic acid, salicin, fish oil (omega-3 fatty acids and specialized, small lipid pro-resolving derivatives), cannabidiol (CBD), tart cherry extract or concentrate, krill oil, astaxanthin, proteolytic enzymes, glucosamine sulfate, chondroitin sulfate, MSM (methyl sulfonylmethane), SAMe (S-adenosylmethionine), and/or triterpenoids.

For the polyphenol, a variety of possible polyphenols are contemplated. Polyphenols include flavonoids, phenolic acids, stilbenes, and lignans. In particular aspects, proanthocyandins may be added to the tributyrin-containing composition. Proanthocyandins include procyanidins, prodelphinidins and propelargonidins which are found in many plants, most notably apples, maritime pine bark and that of most other pine species, cinnamon, aronia fruit, cocoa beans, grape seed, grape skin, some red wines, bilberry, cranberry, pomegranate, black currant, green tea, and black tea. Cocoa beans contain the highest concentrations, and grape seed extract is a readily available source. The amount of proanthocyandin in each single dose may be of between 50-500 mg.

Contemplated compositions may further include extracts from one or more of Acacia catechu, Andrographis paniculata, Scutalleria baicalensis, agmatine sulfate, Stinging Nettle, Sea Buckthorn, curcumin, Cissus Quadrilangularis, Boswellia Serrata, Emu Oil, Arnica, Mangifera indica L. (Anacardiaceae), Lagenaria breviflora, and/or Zingiber officinale (ginger and gingerols/shogaols). Such additional agents may be used in, for example, methods of augmenting and enhancing pain modulation, and/or controlling inflammatory responses. Contemplated extracts suitable for use herein may also provide antioxidant properties, and especially preferred extracts include green tea and/or black tea extracts. While not necessary, it is typically preferred that such extracts will be standardized to a specific component or component class, and particularly to theaflavins, thearubigins, monomeric or polymeric forms of catechins, etc. For example, a black tea extract may standardized to theaflavins.

Still further contemplated compositions may include one or more metabolic enhancers including Hoodia gordonii, yohimbine, synephrine, theobromine, flavonoids, flavanone glycosides such as naringin and hesperidin, chromium (e.g., as picolinate or glycinate, or in association with a complex food matrix), tocopherols, theophylline, alpha-yohimbine, conjugated linoleic acid (CLA), octopamine, evodiamine, passion flower, red pepper, cayenne, raspberry ketone, guggul, green tea, guarana, kola nut, beta-phenethylamines, Acacia rigidula, and/or forskolin (Coleus forskohlli). Such additional ingredients may be used in, for example, methods of enhancing 1) thermogenesis/fat and carbohydrate metabolism; 2) fat loss, weight management and improving body composition (loss of body fat, while retaining or sparing lean body mass/fat free mass/muscle); and/or 3) appetite control/appetite modulation

Yet further contemplated compositions may include anti-fatigue, focusing, and/or energy enhancing ingredients such as creatine, theobromine, theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, tyrosine, N-acetyl-tyrosine, glucuronolactone, taurine, choline, CDP-choline, alpha-GPC, acetyl-L-carnitine, 5-hydroxytryptophan, tryptophan, beta-phenethylamines, Sceletium tortuosum (Mesembrine alkaloids), Dendrobium sp., Acacia rigidula, PQQ (Pyroloquinoline quinone), Ubiquinone(ol), nicotinamide riboside, picamilon, Huperzine A (Chinese clubmoss) or Huperzia serrata, L-dopa, Mucuna pruriens, forskolin (Coleus forskohlli). Such additional ingredients may be used in, for example, methods for enhancing cognitive function, including focus, concentration, sustained attention, working memory, choice and non-choice reaction time, executive function, verbal and non-verbal learning, visuospatial memory and verbal fluency.

With respect to suitable formulations, it is typically preferred that the compositions according to the inventive subject matter are formulated for oral delivery, and all known formulations for oral delivery are deemed suitable for use herein. For example, oral formulations include tablets, dragees, capsules, powders, aqueous or non-aqueous solutions or suspensions, syrup, etc. Most typically, such formulations will include at least one pharmaceutically or nutraceutically acceptable carrier, and are typically prepared to allow administration of a recommended daily dosage in a single dosage unit form. Alternatively, where desired, the dosage unit may also be chosen such that multiple dosage units per day will provide the recommended daily dosage. Alternatively, contemplated compositions may also be included in already known oral formulations. Consequently, contemplated formulations include multi-vitamin preparations and all known preparations are deemed suitable for use herein.

Moreover, contemplated compositions may also be included into an edible carrier to so increase actual or perceived nutritional value of the edible carrier. Most preferably, such edible carrier is in a ready-to-consume format and may be an energy drink, a bottled water product, a carbonated drink, etc., or a snack bar, a cereal, a confectionary item, a plant fiber-containing product etc. In less preferred aspects, parenteral administration is also contemplated and preferably includes injection, transmucosal delivery, and sublingual administration.

### Examples

In a set of experiments, the inventors exposed muscle cells in various in vitro assays where the cells were treated them with a combination of theacrine (commercially available as TeaCrine™), wasabi extract, and copper(I) nicotinate as described in more detail below. All cells were exposed for 3 hr and 24 hr, and cells were analyzed for the following markers: SIRT 1, 4, and 6 as markers for global SIRT activity; TOMM4 as marker for telomere stabilizing complex gene activity; Nrf2 mRNA expression as marker for antioxidant protein status; p27 mRNA expression as marker for cell cycle and apoptosis/autophagy; citrate synthase as marker for mitochondrial volume/capacity; NLRP3 as a marker for inflammasome presence/activity; CDK2, TOMM, ATG mRNA expression as downstream markers for cell cycle, global inflammation, autophagy activation; NAD Metabolome is currently under investigation.

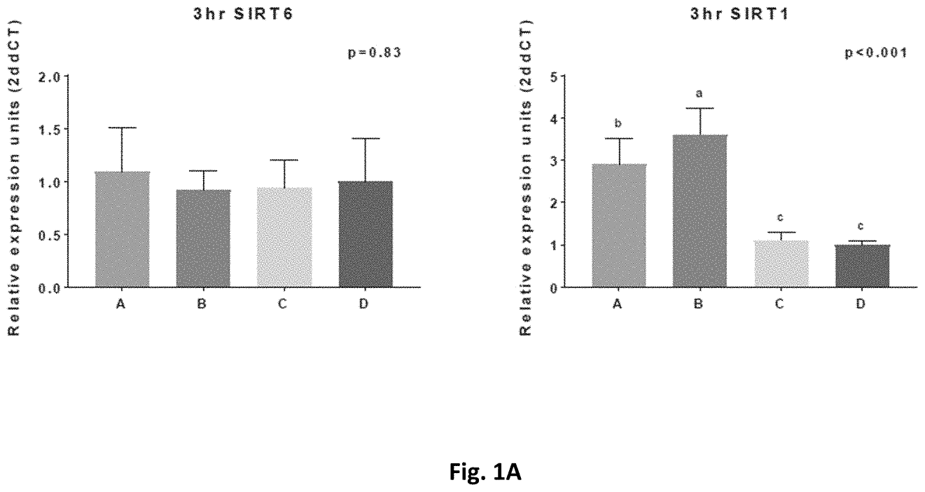

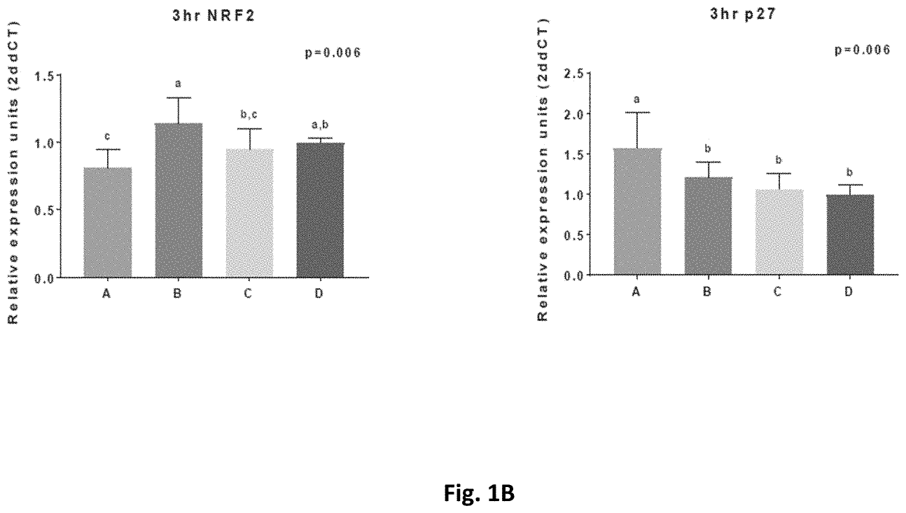

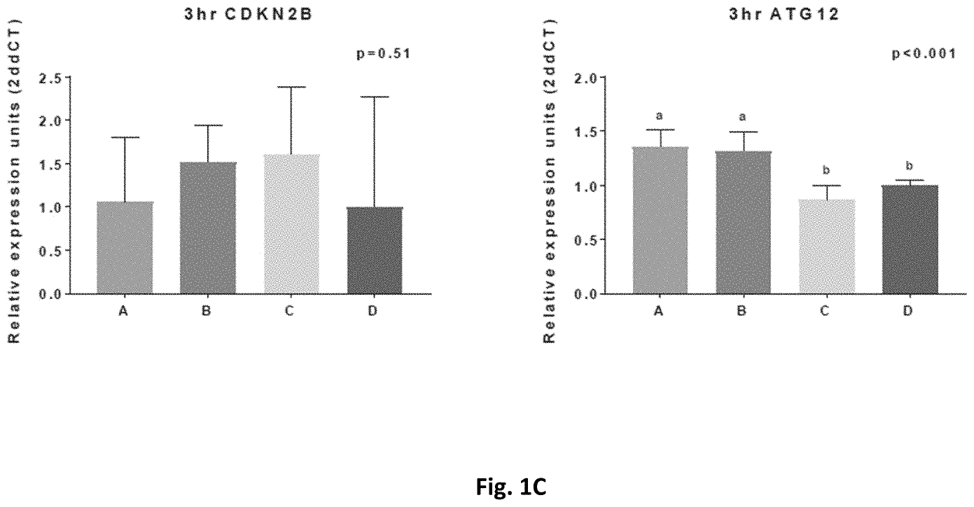

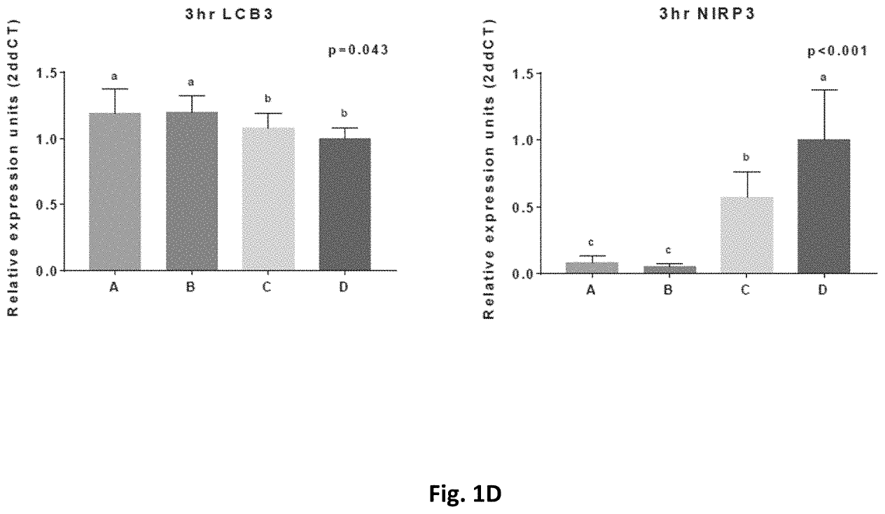

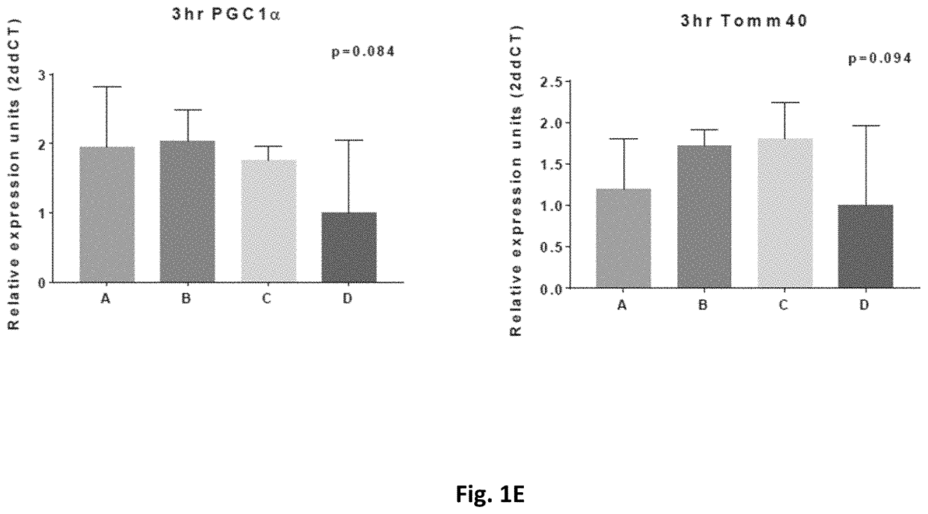

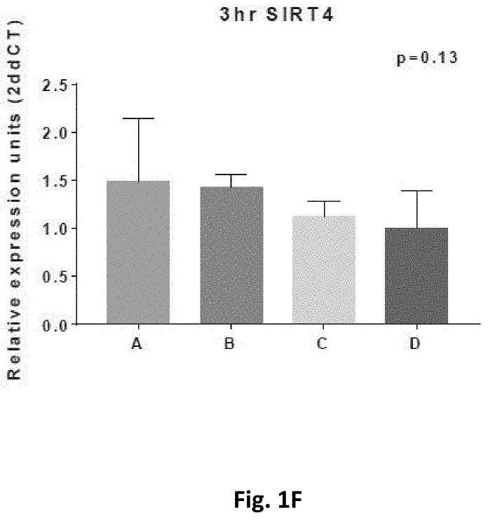

Exemplary Protocols for FIGS. 1A-1F, 2A-2F, 3, and 4:

Passage 6 C2C12 myoblasts, were grown in growth medium (DMEM, 10% FBS, 1% penicillin/streptomycin, and 0.1% gentamycin) on 8 six-well plates at a seeding density of 3×105 under standard culture conditions (37° C. in a 5% CO2 atmosphere). Once myoblast growth reached 80-90% confluency ˜48 h after seeding, differentiation was induced by removing growth medium and replacing it with differentiation medium [DM; DMEM, 2% (vol/vol) horse serum, 1% penicillin/streptomycin, and 0.1% gentamycin]. DM was then replaced every 24 h for 7 days to allow for myotube growth.

Treatment conditions A, B, C and D was mixed into differentiation media and administered to myotubes during differentiation on day 4 for 24 hour treatments (n=6 wells per treatment or 1 plate). The following day treatment conditions A, B, C and D were also administered to myotubes for 3 hours. Hence, this resulted in transient 3-h, acute treatments as well as 24-h, longer-term treatments.

After all treatments, differentiation/treatment media was removed and cells were washed once with phosphate-buffered saline (PBS). Thereafter, PBS was siphoned off and then a subset of cells were scraped from plate and transferred into 250 μL of Trizol for RNA isolation. Following Trizol-based RNA isolation methods, total RNA concentrations were analyzed using a Nanodrop Lite spectrophotometer (Thermo Fisher Scientific), and 1 μg of cDNA were synthesized using a commercial qScript cDNA SuperMix (Quanta Biosciences, Gaithersburg, Md.) per the manufacturer's recommendations. Real-time PCR was performed using gene-specific primers and SYBR green chemistry, and all

PCR reactions were confirmed to produce only one melt product. Relative expression values were performed using the 2-ΔΔCT method where 2-ΔCT [housekeeping gene CT—gene of interest CT] and 2-ΔΔCT (or fold change)=[2-ΔCT value/2-ΔCT average of CTL treatment]. Cyclophilin (CYCLO) was used as a housekeeping gene.

After the RNA scrape described above, 250 μl of ice-cold cell lysis buffer was applied to each well [20 mM Tris.HCl (pH 7.5), 150 mM NaCl, 1 mM Na-EDTA, 1 mM EGTA, 1% Triton, 20 mM sodium pyrophosphate, 25 mM sodium fluoride, 1 mM β-glycerophosphate, 1 mM Na3VO4, and 1 μg/ml leupeptin; Cell Signaling; Danvers, Mass.]. Plates were then scraped and supernatant was removed. Cells were homogenized via micropestles and homogenates were centrifuged at 500 g for 5 min. After centrifugation, insoluble proteins were removed and supernatants containing solubilized cell material were stored at −80° C. This procedure was used in order to perform metabolomics profiling, identify global SIRT activity and quantify citrate synthase activity.

Treatments were grouped in four groups (A-D) and the results are shown in FIGS. 1a-1f. More specifically, Group A is one exemplary combination of TeaCrine™ (Theacrine), a wasabi extract (extract from Wasabia japonica standardized for isothiocynates), and cuprous niacin. Group B is as Group A, but the wasabi extract was replaced with quercetin dihydrate. Group C is nicotinamide riboside (as comparator) at a concentration and active dose 60% ABOVE group A to help demonstrate not just additive, but synergistic benefits as well results above and beyond the main comparator. Group D is silica as negative control with only excipients.

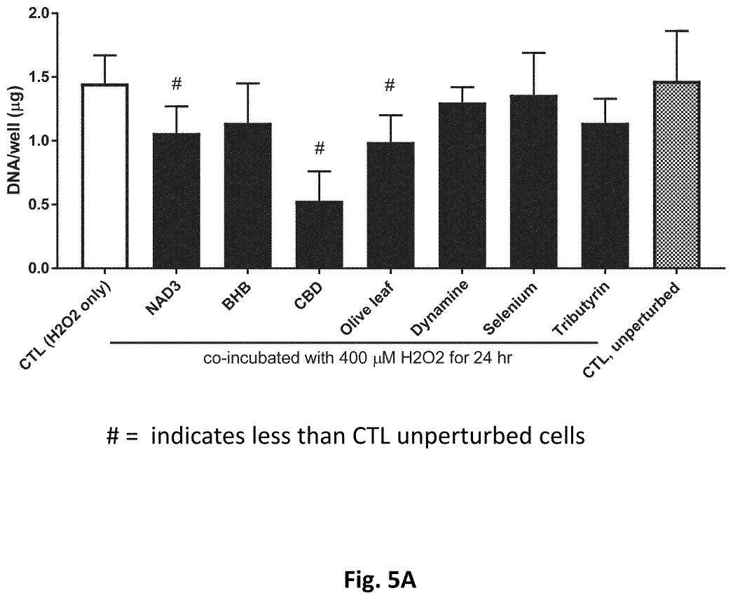

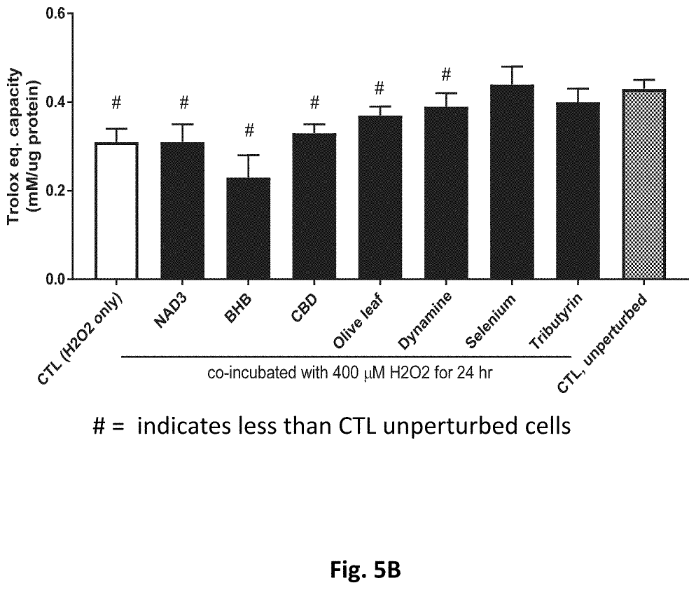

Exemplary Protocols for FIGS. 5A-5B, 6A-6C, and 7A-7C:

Treatments and cells. 6-hour and 24-hour cell culture treatments were with one of the following compounds: 1. Control or “vehicle” (DMSO+phosphate-buffered saline only); 2. Niagen (5 μg/mL); 3. BHB (beta hydroxybutyrate) (30 ug/mL); 4. CBD (cannabidiol) (5 ug/mL); 5. Olive leaf (5 ug/mL); 6. Dynamine (1 ug/mL); 7. Selenium (2 ug/mL); 8. Tributyrin (5 ug/mL)

Treatment materials and methods. C2C12 cells were treated with compounds 1-8 as listed above for 6 hours without hydrogen peroxide (H2O2) (termed “unperturbed cells” throughout); or cells were treated with compounds 1-8 above for 24 hours with a concomitant 400 uM hydrogen peroxide (H2O2).

The C2C12 line resembles mature muscle cells given that they are multinucleated and are past the rapid growth/proliferation phase.

Molecular markers and assays.

Autophagy flux (or activity) was assessed by assaying the LC3II/I ratio. This assay has been deemed the “gold standard” in autophagy monitoring (see e.g., Autophagy, 2007 November-December; 3(6):542-5).

Total antioxidant capacity was assessed using a Trolox equivalent antioxidant capacity (TEAC) assay.

DNA damage was assessed using a phosphorylation assay for H2AX. This assay has been deemed as a commonly assayed biomarker where increases in this marker have been related to increased DNA damage (see e.g., Methods Mol Biol. 2012; 920:613-26).

Oxidative stress was assessed using the 4HNE assay which is a product of cellular lipid oxidation (see e.g., Am J Physiol Cell Physiol. 2016 Oct. 1; 311(4): C537-0543).

Cell viability was assayed measuring DNA content per well where lower values indicate more cell death and decreased cell viability.

Mitochondrial biogenesis was assessed by assaying PGC-1α protein levels; PGC-1α is a master regulator of mitochondrial biogenesis (see e.g., Am J Clin Nutr. 2011 April; 93(4): 884S-890S).

NAMPT protein levels were assessed to examine a key endogenous regulator of the NAD salvage pathway.

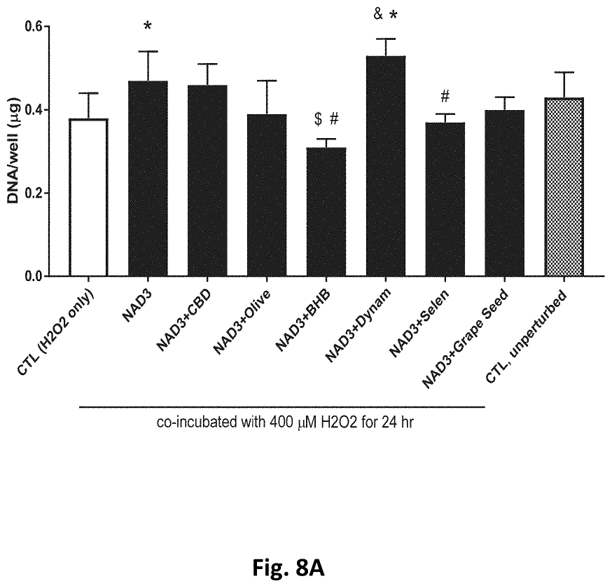

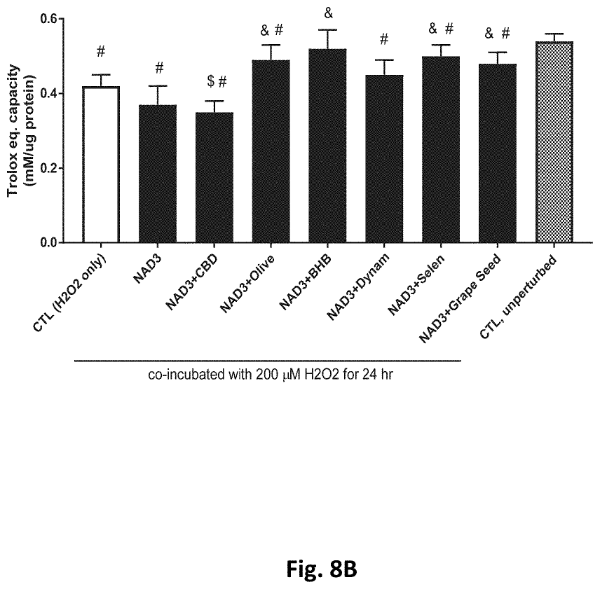

Exemplary Protocols for FIGS. 8A-8B, 9A-9C, and 10A-10D:

Treatments and cells. 6-hour and 24-hour cell culture treatments were with one of the following compounds: 1. Control or “vehicle” (DMSO+phosphate-buffered saline only); 2. NAD3 (5 μg/mL); 3. NAD3 (5 μg/mL) and CBD (cannabidiol) (1.25 ug/mL); 4. NAD3 (5 μg/mL) and Olive leaf (2.5 ug/mL); 5. NAD3 (5 μg/mL) and BHB (beta hydroxybutyrate) (30 ug/mL); 6. NAD3 (5 μg/mL) and Dynamine (1 ug/mL); 7. NAD3 (5 μg/mL) and Selenium (2 ug/mL); 8. NAD3 (5 μg/mL) and Grape Seed Extract (5 ug/mL).

Treatment materials and methods. EOMA (endothelial) cells were treated with compounds 1-8 as listed above for 6 hours without hydrogen peroxide (H2O2) (termed “unperturbed cells” throughout); or cells were treated with compounds 1-8 above for 24 hours with a concomitant 200 uM hydrogen peroxide (H2O2).

The EOMA line resembles vascular endothelial cells.

Molecular markers and assays.

Autophagy flux (or activity) was assessed in EOMA cells by assaying the LC3II/I ratio. This assay has been deemed the “gold standard” in autophagy monitoring (see e.g., Autophagy, 2007 November-December; 3(6):542-5).

Total antioxidant capacity was assessed in the EOMA cells using a Trolox equivalent antioxidant capacity (TEAC) assay.

DNA damage was assessed using a phosphorylation assay for H2AX. This assay has been deemed as a commonly assayed biomarker where increases in this marker have been related to increased DNA damage (see e.g., Methods Mol Biol. 2012; 920:613-26).

Oxidative stress was assessed using the 4HNE assay which is a product of cellular lipid oxidation (see e.g., Am J Physiol Cell Physiol. 2016 Oct. 1; 311(4): C537-0543).

Cell viability was assayed measuring DNA content per well where lower values indicate more cell death and decreased cell viability.

Mitochondrial biogenesis was assessed by assaying PGC-1α protein levels; PGC-1α is a master regulator of mitochondrial biogenesis (see e.g., Am J Clin Nutr. 2011 April; 93(4): 884S-890S).

NAMPT and NLRP3 protein levels were assessed in the EOMA cells to examine a key endogenous regulator of the NAD salvage pathway.

All conditions were statistically compared back to the control condition.

Western blot analysis was performed using antibodies that detected p-H2AX protein, LC3I, LC3II, 4HNE, and NLRP3 proteins in both the C2C12 and EOMA cells. Ponceau stain was used for protein staining.

Exemplary Results:

FIGS. 1A-1F depict exemplary results for mRNA data of SIRT6, SIRT1, NRF2, p27, CDKN2B, ATG12, LCB3, NLRP3, PGC1α, Tomm40, and SIRT4, as indicated, for 3-hour (hr) treatments with Group A, B, C, or D treatments as indicated and as described herein. One-way analysis of variances (ANOVAs) were performed to determine if the treatments significantly differed, and least significant difference (LSD) post hoc tests were performed in order to determine where the significance occurred. Treatments with different superscript letters (a, b, c, or d) indicate treatments differed from each other.

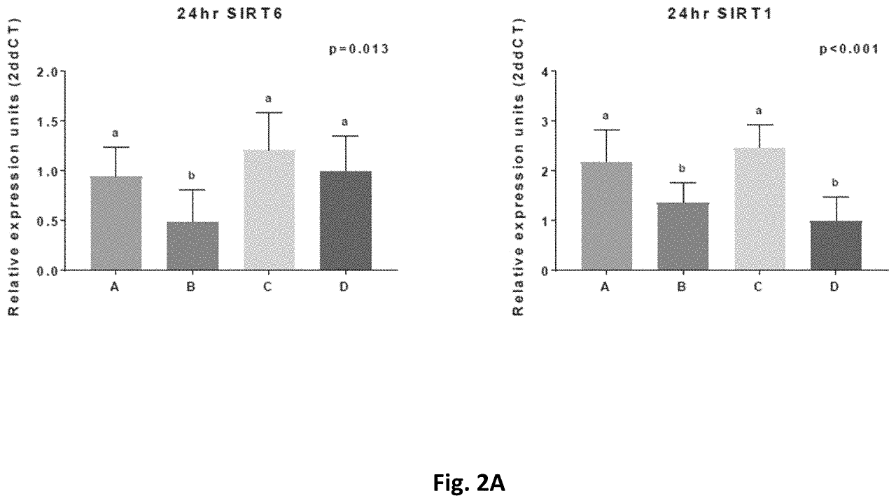

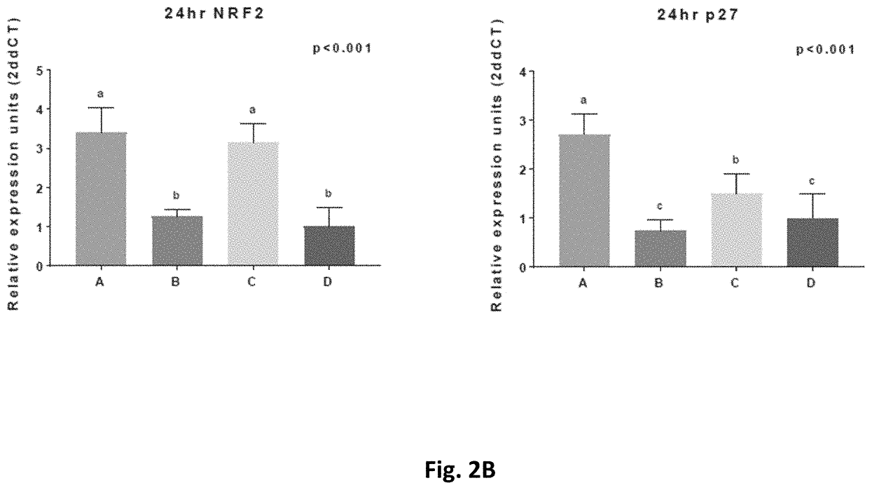

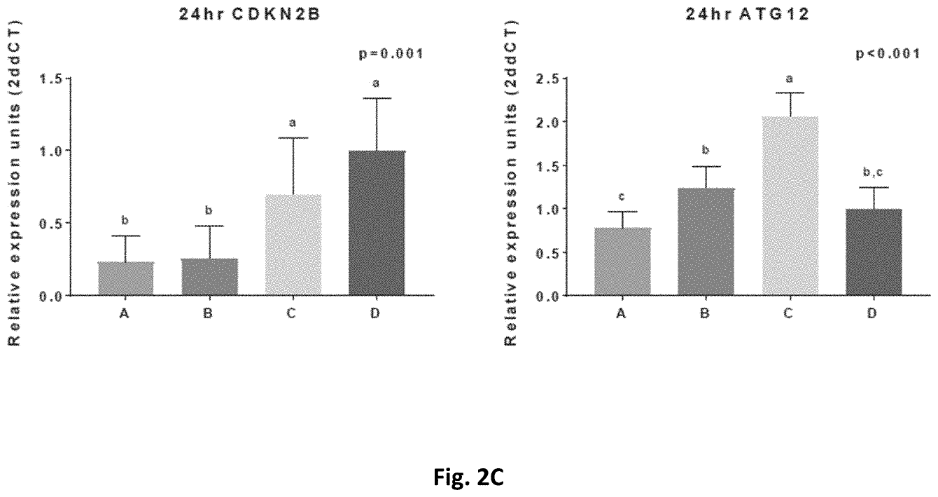

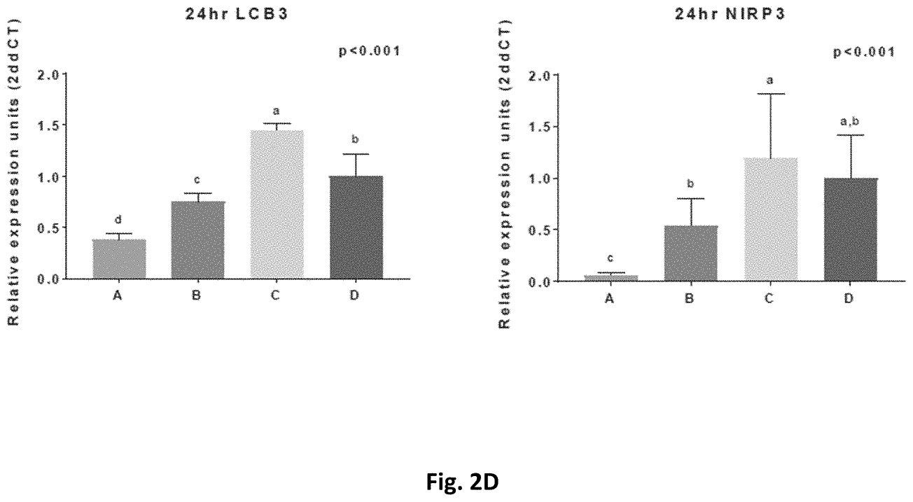

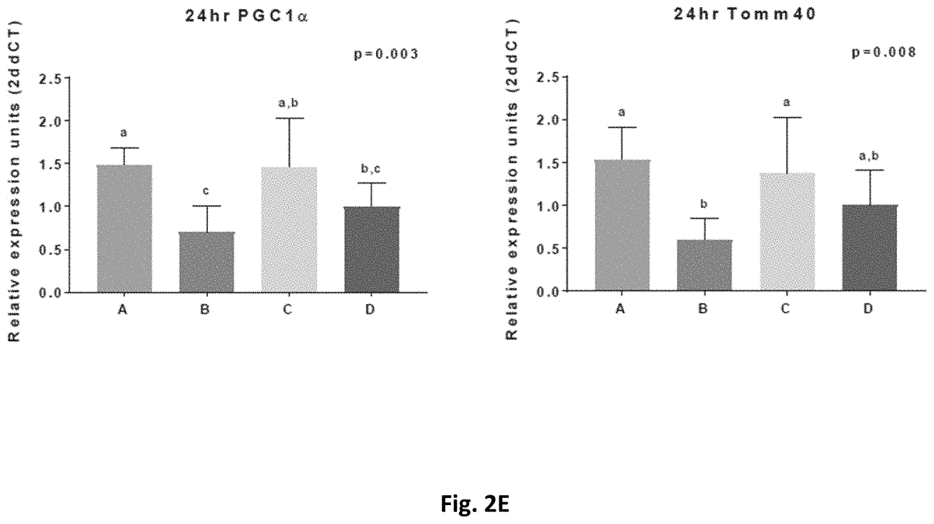

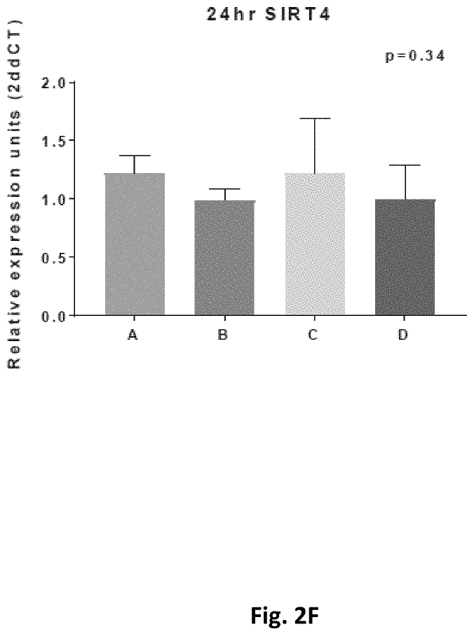

FIGS. 2A-2F depict exemplary results for mRNA data SIRT6, SIRT1, NRF2, p27, CDKN2B, ATG12, LCB3, NLRP3, PGC 1α, Tomm40, and SIRT4, as indicated, for 24-hr treatments with Group A, B, C, or D treatments as indicated and as described herein. One-way ANOVAs were performed to determine if treatments significantly differed, and LSD post hoc tests were performed in order to determine where the significance occurred. Treatments with different superscript letters (a, b, c, or d) indicate treatments differed from each other.

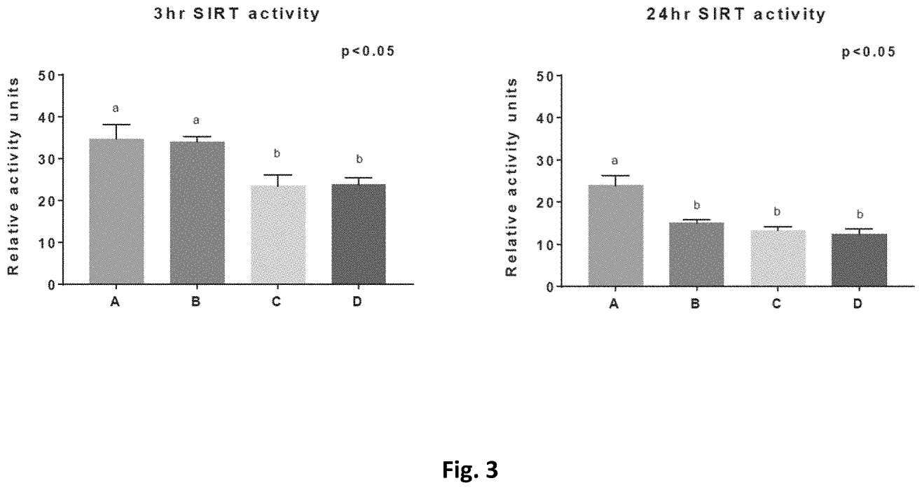

FIG. 3 depicts exemplary results for SIRT activity for 3 hour and 24 hour treatments with Group A, B, C, or D, as indicated and as described herein. One-way ANOVAs were performed to determine if treatments significantly differed, and LSD post hoc tests were performed in order to determine where the significance occurred. Treatments with different superscript letters indicates treatments differed from each other.

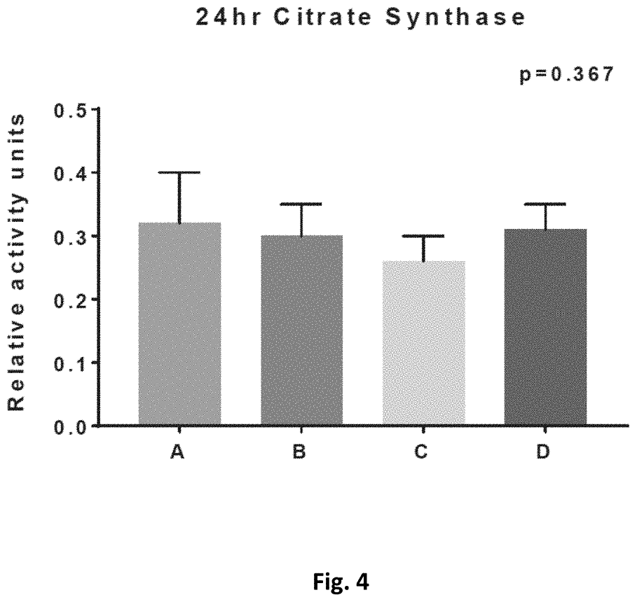

FIG. 4 depicts exemplary results for citrate synthase activity (mitochondrial capacity marker) following 24 h treatments with Group A, B, C, or D, as indicated and as described herein. One-way ANOVAs were performed to determine if treatments significantly differed, but the ANOVA yielded p=0.367.

FIG. 5A depicts exemplary results for cell viability (DNA/well (ug)) for cells incubated without hydrogen peroxide (CTL, unperturbed), cells incubated with hydrogen peroxide (H2O2), and cells co-incubated with NAD3, BHB, CBD, olive leaf, dynamine, selenium, or tributyrin as indicated.

FIG. 5B depicts exemplary results for total antioxidant capacity (Trolox) for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated. As shown, Selenium as well as Tributyrin increase or restore antioxidant capacity in the presence of H2O2.

FIG. 6A depicts exemplary results for autophagy for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

FIG. 6B depicts exemplary results for DNA damage for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

FIG. 6C depicts exemplary results for cellular oxidative stress for cells incubated without hydrogen peroxide (H2O2) (CTL, unperturbed), cells incubated with H2O2, and cells co-incubated with NAD3, BHB, CBD, olive leaf, dynamine, selenium, or tributyrin as indicated.

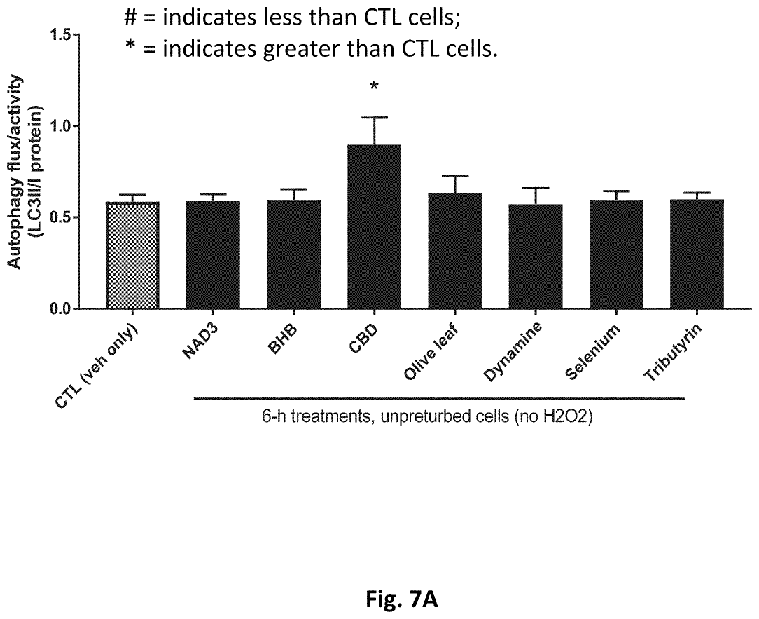

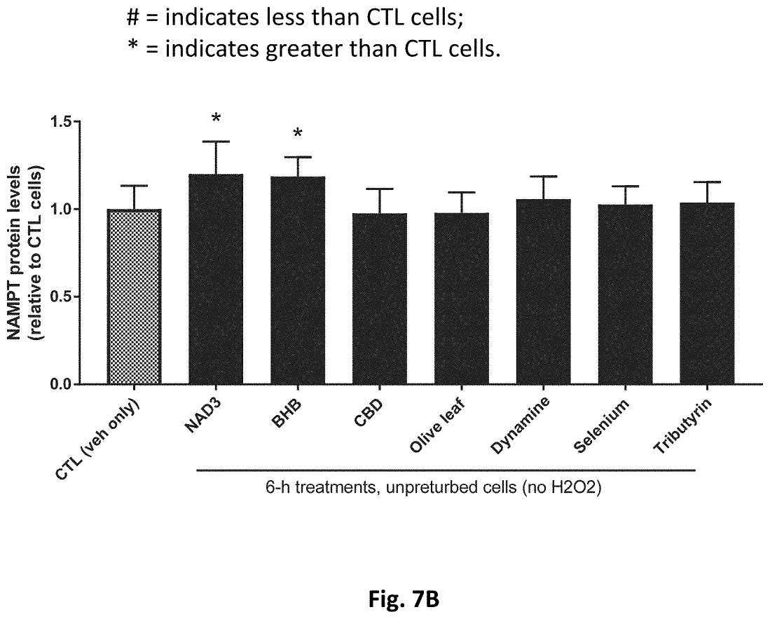

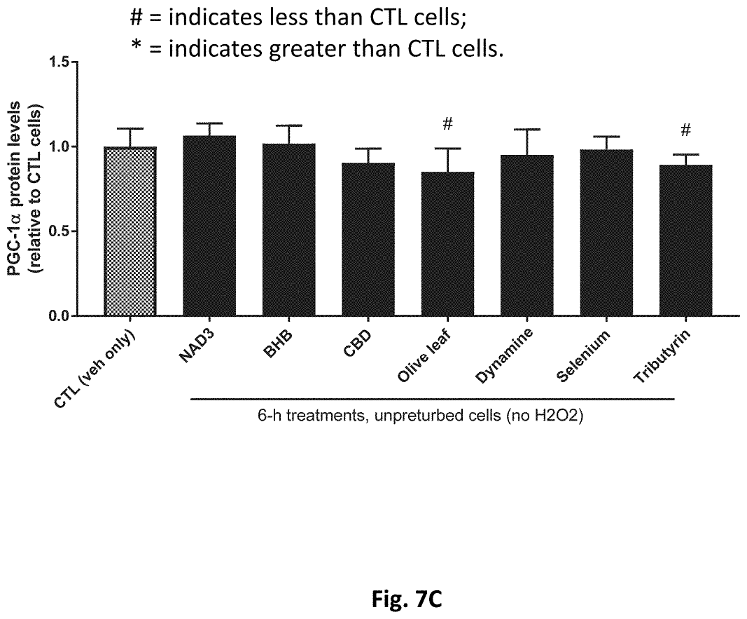

FIG. 7A depicts exemplary results for autophagy for cells incubated for 6 hours without hydrogen peroxide (H2O2) (CTL, vehicle (veh) only), and cells incubated with NAD3, BHB, CBD, olive leaf, dynamine, selenium, or tributyrin as indicated.

FIG. 7B depicts exemplary results for quantifying NAMPT protein levels in cells incubated for 6 hours without hydrogen peroxide (H2O2) (CTL, vehicle (veh) only), and cells incubated with NAD3, BHB, CBD, olive leaf, dynamine, selenium, or tributyrin as indicated.

FIG. 7C depicts exemplary results for quantifying PGC1α protein levels in cells incubated for 6 hours without hydrogen peroxide (H2O2) (CTL, vehicle (veh) only), and cells incubated with NAD3, BHB, CBD, olive leaf, dynamine, selenium, or tributyrin as indicated.

FIG. 8A depicts exemplary results for cell viability (DNA/well (ug)) for cells incubated without hydrogen peroxide (CTL, unperturbed), cells incubated with hydrogen peroxide (H2O2), and cells co-incubated with NAD3, NAD3+ CBD, NAD3+olive leaf, NAD3+BHB, NAD3+dynamine, NAD3+selenium, or NAD3+grape seed, as indicated.

FIG. 8B depicts exemplary results for total antioxidant capacity (Trolox) for cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

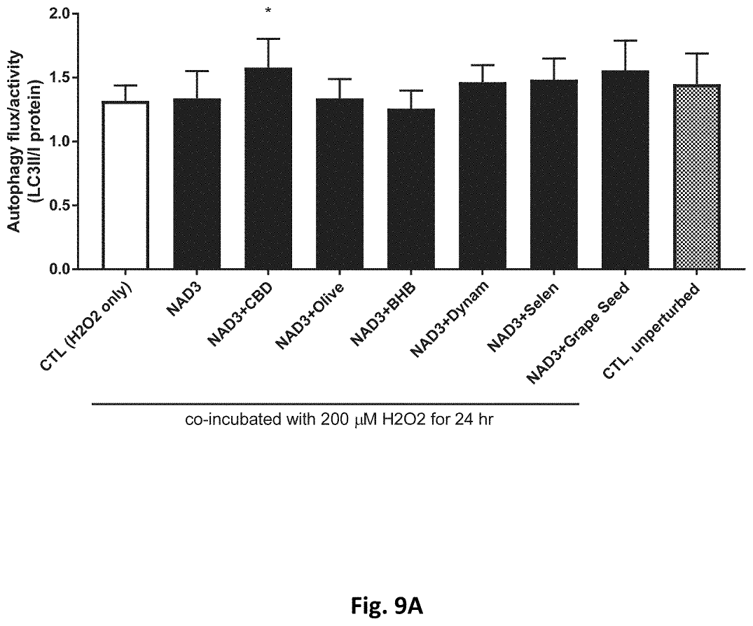

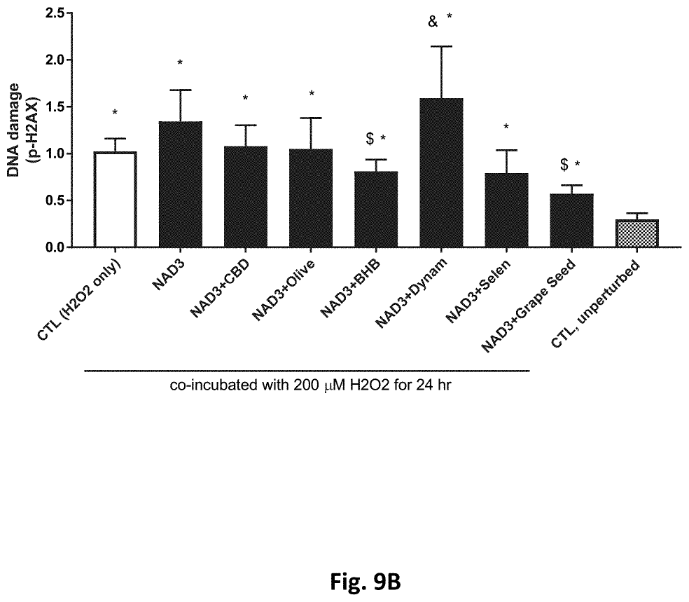

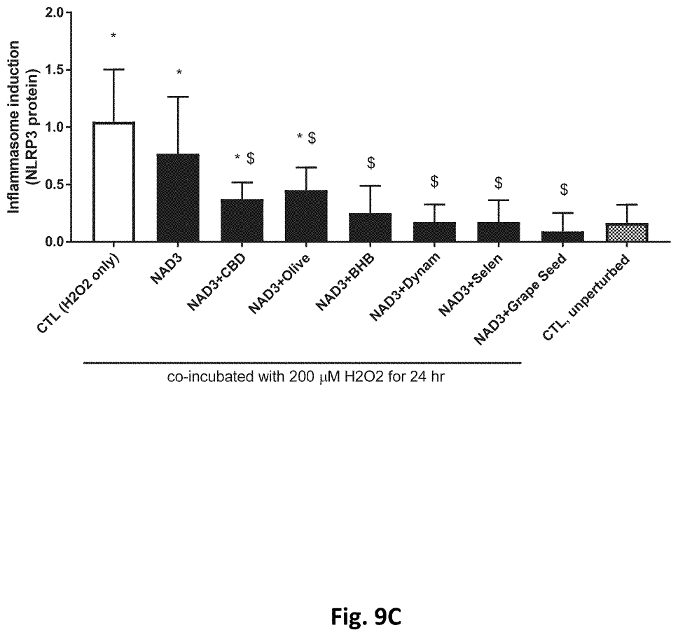

FIG. 9A depicts exemplary results for autophagy for cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

FIG. 9B depicts exemplary results for DNA damage for cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

FIG. 9C depicts exemplary results for quantifying inflammasome induction by measuring the amount of NLRP3 protein in cells incubated without hydrogen peroxide (H2O2) (CTL, unperturbed), cells incubated with H2O2, and cells co-incubated with NAD3, NAD3+ CBD, NAD3+olive leaf, NAD3+BHB, NAD3+dynamine, NAD3+selenium, or NAD3+grape seed, as indicated.

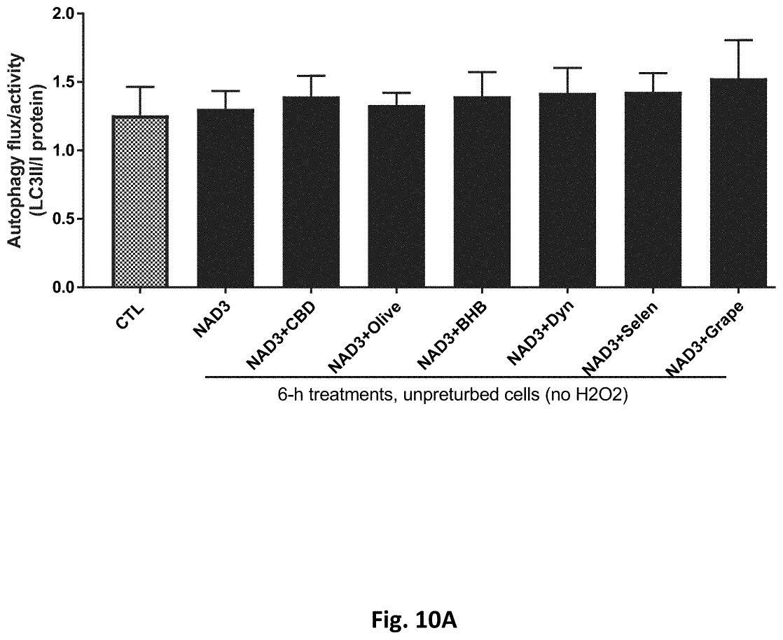

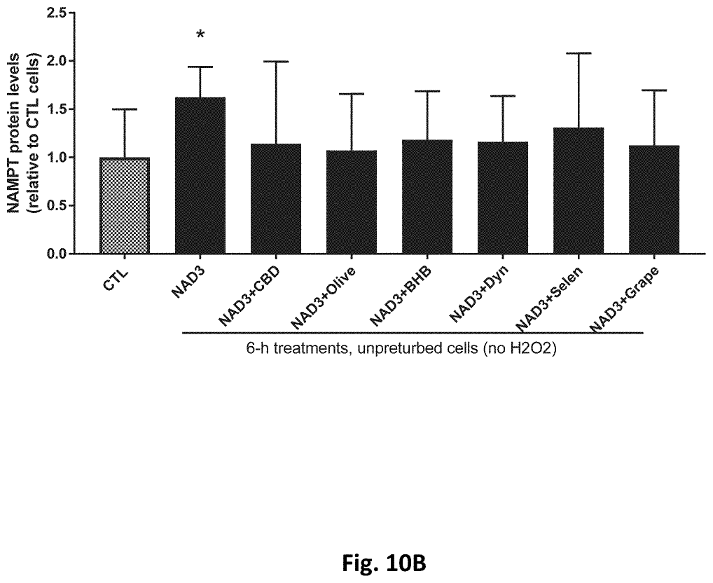

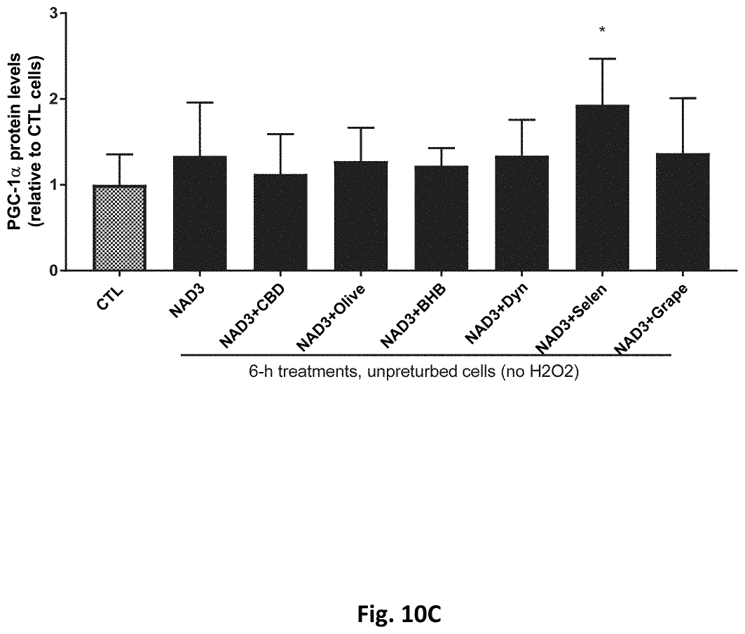

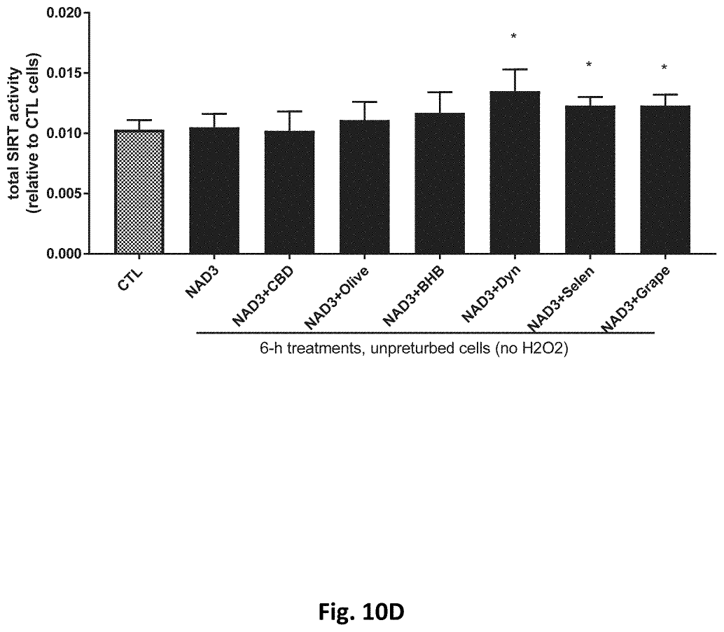

FIG. 10A depicts exemplary results for autophagy for cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 10B depicts exemplary results for quantifying NAMPT protein levels in cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 10C depicts exemplary results for quantifying PGC1α protein levels in cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

FIG. 10D depicts exemplary results for quantifying total SIRT activity in cells incubated for 6 hours without hydrogen peroxide (H2O2) (CTL), and cells incubated with NAD3, NAD3+ CBD, NAD3+olive leaf, NAD3+BHB, NAD3+dynamine, NAD3+selenium, or NAD3+grape seed, as indicated.

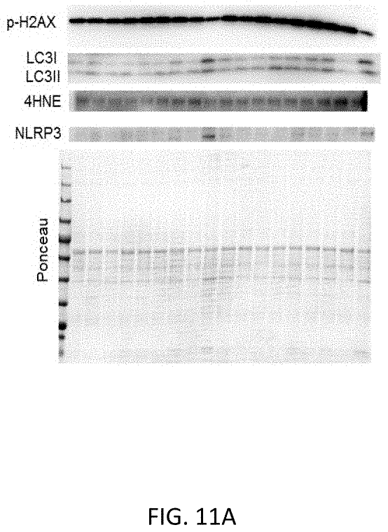

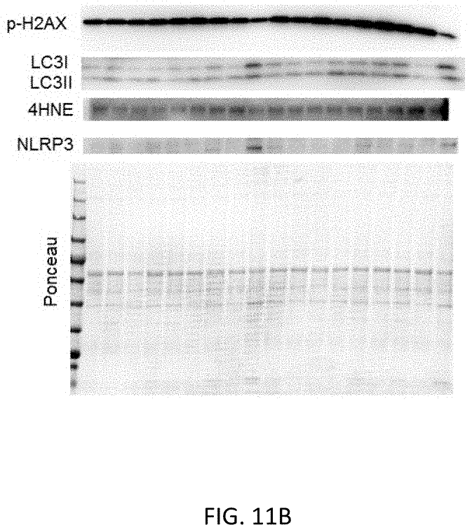

FIG. 11A depicts representative protein gels of C2C12 (muscle cells) with Western blotting analysis shown for p-H2AX, LC3I, LC3II, 4HNE, and NLRP3, and Ponceau protein staining, as indicated.

FIG. 11B depicts representative protein gels of EOMA (endothelial cells) with Western blotting analysis shown for p-H2AX, LC3I, LC3II, 4HNE, and NLRP3, and Ponceau protein staining, as indicated.

As can be readily taken form the results, acute (3 hrs) and longer term (24 hrs) effects on various markers were observed for contemplated combinations. For example, acute differences were, inter alia, observable for SIRT 1, SIRT6, and SIRT4, as well as for p27, PGC1α, and NLRP3, while longer term results were apparent for p27, CDKN2B, LCB3, NLRP3, and TOMM40.

Furthermore, with specific reference to FIG. 5B, tributyrin and selenium were observed to increase (e.g., restore) total antioxidant activity when cells were co-incubated with either tributyrin or selenium and H2O2. Similarly, with reference to FIG. 8B, NAD3 and Olive leaf, NAD3 and BHB, NAD3 and Selenium, and NAD3 and Grape Seed extract all increase total antioxidant capacity over cells treated with H2O2.

Additionally, with specific reference to FIG. 6C, beta hydroxybutyrate (BHB) was observed to decrease cellular oxidative stress in cells co-incubated with H2O2 and BHB. And notably, with reference to FIG. 9C, NAD3 in combination with olive leaf, BHB, dynamine, selenium, and grape seed extract decreased the induction of NLRP3 in the present of H2O2 (oxidative stress).

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A composition for reducing cellular ageing, improving cellular stress resilience, and/or increasing longevity, comprising:
a cytoprotective formulation comprising a combination of (a) theacrine, (b) an isothiocyanate, and (c) a copper-(I)-nicotinate complex;
wherein the cytoprotective formulation is formulated for oral administration with a nutritionally or pharmaceutically acceptable carrier, or for topical administration; and
wherein the theacrine, the isothiocyanate, and the copper-(I)-nicotinate complex are present in the composition in amounts that upregulate gene expression of SIRT1 to thereby reduce cellular ageing, improve cellular stress resilience, and/or increase longevity.

2. The composition of claim 1, wherein the theacrine is present in and provided as a Camellia sp., Theobroma sp., or Coffea sp. extract.

3. (canceled)

4. (canceled)

5. The composition of claim 1, wherein the isothiocyanate is allyl isothiocyanate or 2-phenylethyl isothiocyanate.

6. (canceled)

7. The composition of claim 1, wherein the composition further comprises one or more additional nutritional ingredients.

8. The composition of claim 7, wherein the one or more additional nutritional ingredients include a ketogenic compound.

9. The composition of claim 8, wherein the ketogenic compound is acetoacetate, butyrate, polyhydroxybutyrate (PHB), beta-hydroxybutyrate (BHB), or combinations thereof.

10. The composition of claim 7, wherein the one or more additional nutritional ingredients include a SIRT enhancing agent.

11. The composition of claim 10 wherein the SIRT enhancing agent is fisetin, resveratrol, quercetin, or a catechin.

12. The composition of claim 7, wherein the one or more additional nutritional ingredients includes a NAD enhancing agent.

13. The composition of claim 12, wherein the NAD enhancing agent is niacinamide, nicotinamide mononucleotide (NMN), or niacin.

14. The composition of claim 7, wherein the one or more additional nutritional ingredients include tributyrin.

15. The composition of claim 7, wherein the one or more additional nutritional ingredient include selenium.

16. The composition of claim 7, wherein the one or more additional nutritional ingredients include niacinamide, nicotinamide riboside alternative (NAD3), selenium, olive leaf, a polyphenol, BHB, and/or tributyrin.

17. The composition of claim 16, wherein the polyphenol is grape seed extract.

18.-25. (canceled)

26. The composition of claim 1 formulated for oral administration to a mammal.

27. The composition of claim 1, wherein the isothiocyanate is provided by a wasabi extract.

28. The composition of claim 26, wherein the isothiocyanate is provided by a wasabi extract.
